SEQUENCE LISTING (38 sequences)

SEQ ID NO: 1 - protein, 14 aa, synthetic construct
EMPDAFASPDPDIW

SEQ ID NO: 2 - protein, 29 aa, synthetic construct
VATILTDLASSSSRTTVLLSANLQKEESS

SEQ ID NO: 3 - protein, 12 aa, synthetic construct
ENSSDRISVSRS

SEQ ID NO: 4 - protein, 20 aa, synthetic construct
ALPKPTKPSLRASSYPLGLP

SEQ ID NO: 5 - protein, 5 aa, synthetic construct
GGGGS

SEQ ID NO: 6 - protein, 6 aa, synthetic construct
GGGGGS

SEQ ID NO: 7 - protein, 420 aa, Cutibacterium acnes
KTADPRARAIIAKTDKRVATILTDLASSSSRTTVLLSANLQKEESSFITTTARAISSIACAWATPGSAYHAEPHVLSACIDALKDFCRLRYHPSQDEYGNWWDWEDGASRAIGDVMCILHDALPTDVMAAAAAGIDHFVPDPWYQQPESVKPTAHPTQPVISTGANRMDLTRAVICRSIATGDESKLRHAVQGLPDSWRTVAEGDGFRADGGFIQHSHVPYTGSYGDVLLSGLAMLLPLVAGTRFDITDSAQANLLSQVERGIVPVMYGGQILDCVRGRSISRIDEPAAMHGMSIARSMLLMANAIPAHRAELWRGTVHGWMTRNTFDHLSEPASLRDIDLFDTAANVRPIPESSTPTYFASIDRLVHRTPNWLIAVSNCSNRISWYEYGNSENEWASRTSQGMRYLMLPEDMGQYEDGF

SEQ ID NO: 8 - protein, 420 aa, Cutibacterium acnes
RTSDPRARAIIAKTDRKVAEILTDLVSGSSRQTVLISADLRKEQSPFITKTARAIESMACAWATPGSSYHKDPEILSACIEGLRDFCRLRYNPSQDEYGNWWDWEDGASRATGDEKRLRHAVDGLPDAWRVTTEGDGFRADGGFIQHSHIPYTGGYGDVLFSGLAMLFPLVVADVMCILHDVLPPEVMSAAAAGIDHFIPDPWFQQPASVKPTANPVQPVVSTGANRMDLTRAVMCRSIASGMRFDIVESARKAFHDQVERGFIPVMYNGQILDDVRGRSISRINGSAAMHGISIARAMLMMADALPTHRAEQWRGIVHGWMARNTFDHLSEPSTLVDISLFDAAAKARPVPESSTPSYFASMDRLVHRTADWLITVSNCSDRIAWYEYGNGENEWASRTSQGMRYLLLPGDMGQYEDGY

SEQ ID NO: 9 - protein, 6 aa, synthetic construct
PYQVPS

SEQ ID NO: 10 - protein, 38 aa, synthetic construct
PEMPDAFASPDPDIWSALCEKWTDIITGRNAAKTADPR

SEQ ID NO: 11 - protein, 28 aa, synthetic construct
ATILTDLASSSSRTTVLLSANLQKEESS

SEQ ID NO: 12 - protein, 8 aa, synthetic construct
SAYHAEPH

SEQ ID NO: 13 - protein, 12 aa, synthetic construct
LRYHPSQDEYGN

SEQ ID NO: 14 - protein, 23 aa, synthetic construct
VPDPWYQQPESVKPTAHPTQPVI

SEQ ID NO: 15 - protein, 12 aa, synthetic construct
PDSWRTVAEGDG

SEQ ID NO: 16 - protein, 5 aa, synthetic construct
RFDIT

SEQ ID NO: 17 - protein, 10 aa, synthetic construct
ISRIDEPAAM

SEQ ID NO: 18 - protein, 7 aa, synthetic construct
PAHRAEL

SEQ ID NO: 19 - protein, 34 aa, synthetic construct
TRNTFDHLSEPASLRDIDLFDTAANVRPIPESST

SEQ ID NO: 20 - protein, 8 aa, synthetic construct
GNSENEWA

SEQ ID NO: 21 - protein, 18 aa, synthetic construct
EDMGQYEDGFWATVDYSA

SEQ ID NO: 22 - protein, 22 aa, synthetic construct
TVDSTPLKRAVGTAWAERTPDN

SEQ ID NO: 23 - protein, 6 aa, synthetic construct
KVGKTP

SEQ ID NO: 24 - protein, 10 aa, synthetic construct
TITSKTSFDN

SEQ ID NO: 25 - protein, 23 aa, synthetic construct
EKRKGSWIDVNPARTVKGFNEAI

SEQ ID NO: 26 - protein, 13 aa, synthetic construct
RSQTMALAQRPVD

SEQ ID NO: 27 - protein, 5 aa, synthetic construct
CLLTK

SEQ ID NO: 28 - protein, 5 aa, synthetic construct
TQKRP

SEQ ID NO: 29 - protein, 6 aa, synthetic construct
ADLGGQ

SEQ ID NO: 30 - protein, 285 aa, synthetic construct
EMPDAFASPDPDIWGVATILTDLASSSSRTTVLLSANLQKEESSGENSSDRISVSRSGALPKPTKPSLRASSYPLGLPGSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 31 - protein, 60 aa, synthetic construct
YQVPSRRTFLSLSALSAIAIAASPEMPDAFASPDPDIWSALCEKWTDIITGRNAAKTADP

SEQ ID NO: 32 - protein, 60 aa, synthetic construct
FGTPSRRTFLTASALSAMALAASPTVTDAIAAPGPDSWSALCERWIDIITGRRAARTSDP

SEQ ID NO: 33 - protein, 60 aa, synthetic construct
RARAIIAKTDKRVATILTDLASSSSRTTVLLSANLQKEESSFITTTARAISSIACAWATP

SEQ ID NO: 34 - protein, 60 aa, synthetic construct
RARAIIAKTDRKVAEILTDLVSGSSRQTVLISADLRKEQSPFITKTARAIESMACAWATP

SEQ ID NO: 35 - protein, 60 aa, synthetic construct
TADAPAIIQTQAQGSRVEVIMSEPTQKRPSLTVAIEGVWTVENSSDRISVSRSDKTTTLR

SEQ ID NO: 36 - protein, 60 aa, synthetic construct
AVNRPALVQTRESANQMEVVIVEPTQKRGSLTVTIEGSWKVKTADSHVDVSCENAAGTLH

SEQ ID NO: 37 - protein, 29 aa, synthetic construct
VTLSPALPKPTKPSLRASSYPLGLPHTSS

SEQ ID NO: 38 - protein, 6 aa, synthetic construct
ATGDES

(19) United States
(12) Patent Application Publication
(10) Pub. No.: US 2026/0027194 A1
(43) Pub. Date: Jan. 29, 2026

(54) VACCINES TARGETING C. ACNES HYALURONIDASE FOR PROPHYLAXIS AND TREATMENT OF ACNE VULGARIS

(71) Applicants: Irshad Ahmed Hajam, San Diego, CA (US); Cedars-Sinai Medical Center, Los Angeles, CA (US); The Regents of the University of California, Oakland, CA (US)

(72) Inventors: Irshad Ahmed Hajam, San Diego, CA (US); George Y. Liu, San Diego, CA (US); Ramachandran Murali, Beverly Hills, CA (US); Stacey Kolar, Los Angeles, CA (US)

(73) Assignees: Cedars-Sinai Medical Center, Los Angeles, CA (US); The Regents of the University of California, Oakland, CA (US)

(21) Appl. No.: 19/144,225

(22) PCT Filed: Dec. 29, 2023
(86) PCT No.: PCT/US2023/086413

§ 371 Date: Jun. 27, 2025

Related U.S. Application Data
(60) Provisional application No. 63/436,332, filed on Dec. 30, 2022.

(51) Int. Cl. A61K 39/05; A61K 39/00; A61P 17/10; C12N 9/88

(52) U.S. Cl. CPC A61K 39/05; A61P 17/10; C12N 9/88; C12Y 402/02001; A61K 2039/53; A61K 2039/55522; A61K 2039/55544; A61K 2039/55561; A61K 2039/55566; A61K 2039/55572; A61K 2039/55577

(57) ABSTRACT

The present invention provides for immunogenic peptides and compositions for the treatment of acne, or the reduction of the likelihood of having acne in a subject in need thereof.

### CROSS-REFERENCE TO RELATED APPLICATIONS

This application includes a claim of priority under 35 U.S.C. § 119(e) to U.S. provisional patent application No. 63/436,332, filed Dec. 30, 2022, the entirety of which is hereby incorporated by reference.

### STATEMENT REGARDING FEDERALLY SPONSORED RESEARCH OR DEVELOPMENT

This invention was made with Government support under Grant Nos. AI141401 and AI138053 awarded by the National Institutes of Health. The Government has certain rights in the invention.

### REFERENCE TO SEQUENCE LISTING

This application contains a Sequence Listing submitted as a computer readable form named “SequenceListing_065472_000903WOPT.xml”, having a size in bytes of 35,558 bytes, and created on Dec. 29, 2023. The information contained in this computer readable form is hereby incorporated by reference in its entirety.

### FIELD OF INVENTION

This invention relates to the treatment and prophylaxis of acne.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Acne vulgaris affects four of five individuals sometime during their lifetime. Predisposition to acne is dependent on both host and environmental factors. Among these, the contribution of skin commensal C. acnes has been debated since healthy and acne-prone subjects are robustly colonized with C. acnes. More careful characterization of C. acnes isolates directly from acne lesions demonstrates the importance of C. acnes genetic elements as a major acne determinant, as development and severity of acne is clearly C. acnes strain and phylotype dependent. Accordingly, C. acnes strains have been categorized based on their health or acne association. Subsequent metagenomics studies unveiled sets of genes that are prominently present in acne- or health-associated strains of C. acnes, thereby ushering in a new potential front in the quest for the understanding of acne pathogenesis. Yet, acne pathogenesis is poorly understood, hampered by the absence of a robust animal model and poor survival of C. acnes in rodents.

Accordingly, there remains a need for a better understanding of C. acnes and an unmet need for the prevention and treatment of C. acnes.

### SUMMARY OF THE INVENTION

The following embodiments and aspects thereof are described and illustrated in conjunction with compositions and methods which are meant to be exemplary and illustrative, not limiting in scope.

Various embodiments of the present invention provide an immunogenic polypeptide, comprising: a fragment of HylA.

In various embodiments, the fragment of HylA can be linked or fused to an adjuvant

In various embodiments, the fragment of HylA can comprise one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the adjuvant can be a tetanus protein, pertussis toxoid, diphtheria toxoid, cytokine, or a fragment thereof.

Various embodiments provide for an mRNA molecule encoding the immunogenic polypeptide of the present invention as described herein.

Various embodiments provide for a polypeptide, comprising: one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the one or more peptides can be linked or fused to an adjuvant.

In various embodiments, the adjuvant can be a tetanus protein, pertussis toxoid, diphtheria toxoid, cytokine, or a fragment thereof.

In various embodiments, the polypeptide can further comprise a linker between the one or more peptides and the adjuvant.

In various embodiments, the polypeptide can further comprise a linker between the one or more peptides and C-terminus of the adjuvant.

In various embodiments, the polypeptide can comprise at least two peptides and further comprising a linker between each of the at least two peptides.

In various embodiments, the linker can be G, polyserine, polyglycine, glycine-serine, GGGGS (SEQ ID NO:5), GGGGGS (SEQ ID NO:6), leucine zipper, r aliphatic, or helical peptides.

Various embodiments provide for a composition comprising a polypeptide of the present invention, and an adjuvant.

In various embodiments, the adjuvant can be alum, hydroxyphosphate sulfate, CpG1018, monophosphoryl lipid A, oil-in-water emulsion, CpG, or QS-21 saponin.

Various embodiments provide for an mRNA molecule encoding a polypeptide of the present invention as described herein.

Various embodiments provide for a method of treating acne, comprising: administering a polypeptide of the present invention or a composition of the present invention, or a composition comprising the mRNA molecule of the present invention, to a subject in need thereof.

Various embodiments provide for a method of reducing the likelihood of acne, comprising: administering a polypeptide of the present invention or a composition of the present invention, or a composition comprising the mRNA molecule of the present invention, to a subject in need thereof.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 (panels a-j) shows that HylA enzyme is a major virulence factor in acne pathogenesis. A, Pie chart showing health- and acne-associated C. acnes phylotypes and association with hylA or hylB gene. B-f, CD1 mice (n=10) were infected intradermally (i.d.) with 2×10⁷ CFU WT (HL043PA1 or HL110PA3) or isogenic mutant (ΔhylA or ΔhylB) C. acnes, followed by topical application of sebum daily. Bacterial burden (b), disease score (c), and cytokines (d-f) at 2 d post-infection. G-i, CD1 mice (n=10) were infected as above with either HL043PA1, ΔhylA or ΔhylA plus recombinant I HylA protein (10 μg). Disease score (g), and tissue cytokines (h,i) at 2d post-infection. B-i, Data were from two independent experiments with each data point representing one mouse. Bars denote median. j, tissue cytokines at 2d post-infection. The data in j were analyzed by one-way ANOVA with Tukey's post-hoc test. The data in b,c and e-h were analyzed by one-way ANOVA with Tukey's post-hoc test. The data in d and i were analyzed by non-parametric Kruskal-Wallis one-way ANOVA test.

FIG. 2 (panels a-e) shows the HA degradation and structural features of HylA and HylB enzymes. A,b, HPLC profile of HMW HA (2 mg/ml) digested for 24 hr with rHylA or rHylB (1 ug). Digested HA peaks (HA-2−, 4 and 6) were quantified using known concentrations of purified HA oligosaccharides (see FIGS. 10 and 11). Larger sized HA fragments, highlighted with a green circle, are visualized only with recombinant HylA (rHylA) digested HA. The results are representative of at least 2 independent experiments. C, comparison of HylA and HylB crystal structures. HylA and HylB are shown by cartoon in magenta and orange, respectively. The structural domains and linker are labeled. D, the active site cleft components and comparison of electrostatic potentials of solvent excluded surface. The electrostatic surface view is shown for the HylA and HylB crystal structures. The electrostatic potentials of solvent excluded surfaces of Hylase crystal structures were calculated by APBS in PyMol. The active-site cleft is shown by a dashed oval. Red and blue correspond to potentials of −5 kT e⁻¹ and 5 kT e⁻¹, respectively. E, the residue wise similarities and differences at active site cleft of HylA and HylB. The residues are highlighted at different structural components of the cleft and labeled for their functional roles. The HA-6 ligand is taken from the Streptococcus pneumoniae Hyl (SpHyl) crystal structure (PDB: 1LOH) is modelled in HylA active site cleft.

FIG. 3 (panels a-f) shows the comparison of HylA and HylB with bacterial and animal Hyl. A, superimposition of HylA crystal structure with Hyl from Streptomyces coelicolor (ScHyl). B, Comparison of HylA crystal structure with Hyl from Streptococcus pneumoniae (SpnHyl) and Streptococcus agalactiae (SaHyl). HylA, ScHyl, SpnHyl and SaHyl are shown by cartoon representation in magenta, salmon red, green, and cyan, respectively. The PDB IDs for ScHyl, SpnHyl and SaHyl are 2X03, 2BRW and 1F1S, respectively. The dashed circle in panel a and b shows the region of HylA's β-domain whose topology is similar to or different from the homolog. The structural domains and linker are labeled. C, the conformations of the active site cleft are shown. The relative positions of (i.e., the distances between) the LI and/or LII loops from the α-domain and the LIV and/or LV loops from the β-domain defines the open/closed conformation of the Hyl cleft. It is denoted by the black arrows. The Hyl enzyme's cleft from different bacteria, including the crystal structures HylA, HylB, 2X03, 2WCO, 2BRW, 1LOH, 1F1S, and 1LXM are shown by magenta, orange, salmon red, slate blue, green, splitpea green, cyan, and grey, respectively. D, the catalytic tetrad (Tyr-His-Arg-Glu) and residues involving in the neutralization of the substrate's acid (Asx) are shown. The corresponding residues from HylA, HylB, ScHyl, SpnHyl and SaHyl are shown by sticks in magenta, orange, salmon red, green, and cyan, respectively. The HA-6 ligand is taken from the SpnHyl crystal structure (PDB: 1LOH). E, The structure of human Hyl (hHyl1, human hyaluronidase 1) (PDB: 2PE4). The structural organization of hHyl1 is shown as representative of animal hyaluronidases. F, HylA and HylB structural elements that define the catalytic cleft are shown in cartoon representation. HylA and HylB are shown in magenta and orange, respectively. The HA-6 ligand is taken from the SpnHyl crystal structure (PDB: 1LOH) and is shown by sticks in yellow.

FIG. 4 (panels a-h) shows the enzymatic activity of HylA mutants with single amino acid substitutions. A, Position of amino acids residues on HylA crystal that were mutated to corresponding HylB residues. B-h, HPLC profile of HMW HA after 24 hr coincubation with WT or mutant HylA (1 μg): undigested (b), rHylB (c), rHylA (d) or rHylA with single amino acid substitutions (e, h). Quantification of HA digested peaks was performed using known concentrations of purified HA oligosaccharides (see FIGS. 10 and 11). Asterisk (*) represents non-specific peaks present in water control (see FIG. 7). Data are representative of two independent experiments.

FIG. 5 (panels a-f) shows the proinflammatory properties and TLR2 dependence of Hyl degradation products. A, HaCaT cells were stimulated with the HA digested with either supernatant from HylA+HL043PA1 or HylB+HL110PA3. IL-6 in the culture supernatant was measured. B-d, WT, TLR2^{-/-} and TLR4^{-/-} mice were infected i.d. with WT or isogenic ΔhylA HL043PA1 (2×10⁷ CFU) as above. Disease score (b) and skin cytokines (c,d) at 24 hr post-infection. E, IL-6 in WT or TLR2-BMDM culture supernatant after stimulation with rHylA or rHylB digested HA. F, IL-6 in HaCaT cell culture supernatant after stimulation with HA that has been digested with WT or mutant rHylA. Data in a,e,f, are presented as mean±SD and each data point is a technical replicate. B-d, Bars denote median, and each data point represents one individual mouse (n=5-11 for TLR4^{-/-}, TLR2^{-/-} or WT mice infected with HL043PA1, and n=4-5 for TLR4^{-/-}, TLR2^{-/-} or WT mice infected with isogenic ΔhylA). a,e,f, The data are representative of two independent experiments. The p values in a,f were calculated by one-way ANOVA with Tukey's post-hoc test. The p-values in b-d were calculated by non-parametric Mann-Whitney T test.

FIG. 6 (panels a-d) shows that vaccination against HylA improves acne lesions and mitigates inflammation. A-d, mice (n=10) were immunized intraperitoneally (i.p.) with alum plus C-terminus of tetanus protein (TT) or multiple HylA epitopes linked to TT (TT-mHylA), then challenged i.d. with HylA HL043PA1 C. acnes strain. Disease score (a) bacterial burden (b), IL-1b (c) at 48 hr. d, serum anti-HylA or anti-HylB antibody titer after the third immunization with TT-mHylA. Data are from three (a-d) independent experiments with each data point representing one mouse. The data in a-c were analyzed by two-tailed un-paired non-parametric Mann-Whitney Student's/test, and in d by non-parametric Kruskal-Wallis one-way ANOVA test.

FIG. 7 (panels a-h) shows the verification of ΔhylA and ΔhylB enzymatic activity. A, WT and Δhyl culture supernatants were tested for enzymatic activity against HMW HA substrate. Arrows show areas of HA clearance from incubation with WT strains, absent on plates with Δhyl. B-e, HMW HA substrate (2 mg/ml) was digested for 24 hr with supernatant (10 μl) from WT HL110PA3 or HL043PA1 (b,d), or from isogenic ΔhylB or ΔhylA (c,e) and analyzed by HPLC. F, HA digested peaks were quantified using known concentrations of purified HA oligosaccharides (HA-2, HA-4 and HA-6). G,h, Controls undigested HA (g) and water (h). Asterisk (*) represents non-specific peaks present in water control. Green circle (f) shows larger oligomers present only in HMW HA digested with the HL043PA1 supernatant. The results are representative of at least two independent experiments.

FIG. 8 (panels a-c) shows HA disaccharide from HylB digest analyzed by LC-MS in negative ionization mode. A, Extracted ion-chromatogram of elution time of disaccharide (12.12 min). b, Mass (m/z) of the corresponding disaccharide in negative mode (M-H)−. C, Extracted ion chromatogram of HA-tetrasaccharide.

FIG. 9 (panels a-h) shows that HylA enzyme is a major virulence factor in acne pathogenesis. A, CD1 mice (n=10) were infected i.d. with WT (HL043PA1 or HL110PA3) or isogenic mutant (ΔhylA or ΔhylB) C. acnes (2×10⁷ cfu). Representative images of skin lesions 2 d post-infection. B,c, CD1 mice (n=10) were infected as above with HL043PA1, ΔhylA or ΔhylA plus recombinant I HylA protein (10 μg). CFU (b) and IL-1B (c) from skin lesions at 2d post-infection. (d) IL-10 level (n=6 for HL110PA3 and n=7 for ΔHylB). E-g, CD1 mice (n=3 for sebum and PBS, and n=5 for HL043PA1+sebum) were infected i.d. with HL043P1 plus topically applied sebum or topically applied sebum only or no treatment, and then disease score I and cytokines (f-h) were measured on d2 (48 hr). Bars denote median and data were from one to two independent experiments. Data were analyzed by non-parametric two-tailed Mann-Whitney U test (d) or by one-way ANOVA with Tukey's post-hoc test (e-h).

FIG. 10 (panels a-g) shows the kinetics of recombinant Hyl digestion of HA. A-d, HPLC profiles of HMW HA (2 mg/ml) digested with of rHylB (1 μg) for 0-60 min. e, HPLC profile of HMW HA (2 mg/ml) digested with 1 μg of rHylA enzyme for 60 min. f, water control. G, HA peaks were quantified using known concentrations of purified HA oligosaccharides. Oval circle I shows larger oligomers present only in rHylA digested HA. The results are representative of at least two independent experiments.

FIG. 11 (panels a-f) shows the kinetics of HA digestion with supernatant from either HylA or HylB expressing C. acnes strains. A-d, HMW HA substrate (2 mg/ml) was digested with supernatant (10 μl) from either HylA⁺HL043PA1 or HylB⁺HL110PA3 for 1 hr (a,c) or 24 hr (b,d), followed by HPLC analysis. E, Digested HA peaks (DP2, DP4 and DP6) were quantified using known concentrations of purified HA oligosaccharides. F, water control. Oval circle (b) shows larger oligomers present only in HA digested with the HylA⁺HL043PA1 supernatant. The results are representative of at least two independent experiments.

FIG. 12. Hyl-A and Hyl-B protein level in health- and acne-associated C. acnes strains. Two acne-(HL043PA1 and HL043PA2) and two health-(HL110PA3 and HL110PA4) associated strains were grown for 4 days, and supernatants were analyzed by SDS-PAGE for Hyl expression with rHylA and rHylB proteins as positive controls. The results are representative of at least two independent experiments. The yellow arrows indicate expressed HylA and HylB.

FIG. 13 Enzyme activity of HylA and HylB mutations. The enzyme activity profiles for the single amino acid substitutions (point mutations) made in the HylA and HylB enzymes are shown in the graph.

FIG. 14 (panels A-B) show proposed HylA residues affecting/altering the HA-degradation mechanism and phenotype. HylA/B structural elements proposed to be involved in domain motions (Mello et al., 2002). The structural elements are shown in cartoon representation and labeled by L (Loop) and H (Helix) followed by the number. HylA and HylB are shown in magenta and orange, respectively. The hexasaccharide (HA-6) substrate is taken from the SpHyl crystal structure (PDB: 1LOH) and is shown by sticks in yellow. The crucial residues from the loop LIV involving in the cleft opening/closing motion are shown by sticks. Point mutation in one of these residues, S452G, is observed to significantly affect the phenotype. (B) The differences in these residues between HylA and HylB are also highlighted in the sequence alignment. HylA sequence (SEQ ID NO:7); HylA sequence (SEQ ID NO:8)

FIG. 15 (panels a-e) shows HA degradation product from HA incubation with rHylA or single amino acid mutants of rHylA. A-b, HPLC profiles of HMW HA (2 mg/ml) digested for 24 hr with WT (a) or mutant rHylA (b) (1 μg). c, Quantification of HA digested peaks using known concentrations of purified HA oligosaccharides. D,e, Undigested HA (d) and water I served as negative controls. Oval circle (a-b) shows larger oligomers. The results are from one experiment. Asterisk (*) represents non-specific peaks present in water control.

FIG. 16 (panels a-e) a,b, HaCaT cells were stimulated with HA have been predigested with rHylA or rHylB. IL-6 (a) and IL-8 (b) in the culture supernatants. C-d, C57Bl/6 mouse BMDMs were stimulated with rHylA- or rHylB-digested HA. IL-6 (c) and TNF-α (d) in the culture supernatants. E,f, C57Bl/6 WT, TLR2^{-/-} or TLR4^{-/-} mice were infected (2×10⁷ CFU) i.d. with HL043PA1 or isogenic ΔhylA. Bacterial burden I and TNF-α (f) after 24 hr. Bar denotes mean±SD. Data are representative of at least two experiments. Bars denote median (d,e). Each data point represents one mouse (n=5-11 for WT, TLR4^{-/-} or TLR2^{-/-} mice infected with HL043PA1, and n=4-5 for WT, TLR4^{-/-}, or TLR2^{-/-} mice infected with ΔhylA). Data were analysis by one-way Anova with Tukey's post-hoc (a-d) and non-parametric Mann-Whitney T test (e,f).

FIG. 17 rHylA vaccination protects mice against acne. A, Schematic of rHylA immunization followed by C. acnes challenge. B, Serum anti-HylA and anti-HylB antibody titers after the third rHylA immunization. C-f, CD1 mice (n=10) immunized with Alum (Mock) or Alum-rHylA (rHylA) were challenged i.d. with HL043PA1. Disease score I, Bacterial burden (d), IL-6 I and IL-1b (f). g, Titers of serum anti-HylA and anti-HylB from CD1 mice (n=5) immunized with rHylB. H-j, mice were vaccinated with mock, rHylA, or rHylB and then challenged i.d. with HL110PA3. Disease score (h), bacterial burden (i) and IL-1B (j) at day 2 post-infection. Bars denote median. Data are from two independent experiments (b-f) or from one experiment (g-j) with each data point representing one mouse. The data from c-f were analysis by two-tailed un-paired non-parametric Mann-Whitney Student's t test. The data from b,h-j were analyzed by one-way Anova with Tukey's post-hoc test.

FIG. 18 (panels a-d) shows a design of HylA multi-epitope vaccine. A,b, Linear B cell epitopes in HylA protein were predicted using Bepipred Linear Epitope Prediction 2.0 (IEDB analysis resources, tools.iedb.org/bcell/). The chart shows immunogenic peptides with scores above 0.5 (a) and table shows list of predicted peptides (b). c, Alignment of 4 selected predicted peptides (underlined) shows no sequence homology with HylB. D, The 4 predicted epitopes (underlined) were physically linked to the C-terminus of tetanus toxoid protein (italicized). A linker amino acid glycine (G) was placed between each peptide and the C-terminus of tetanus.

FIG. 19 Effect of TT-mHylA vaccination on disease induce by HL043PA1 and HL110PA3 infection. A-b, mice were vaccinated, as in Extended data FIG. 12, with either TT or TT-mHylA and challenged 7 days after vaccination with HL043PA1. TNF-α (a) and IL-6 (b) at 48 hr post-infection. C-f, mice were vaccinated, as above with either TT or TT-mHylA and challenged 7 days after vaccination with HL110PA3. Disease score (c), IL-1ß (d) IL-6, I and TNF-α (f) at 48 hr post-infection. Bars denote median. Data are from three independent (a-b) or one experiment (c-f), with each data point representing one mouse. The p values in a-f were calculated by two-tailed un-paired non-parametric Mann-Whitney Student's t test.

FIG. 20 (panels a-i) shows enzymatic activity of HylA mutants with single amino acid substitutions. A, position of the amino acid residues (shown by sticks in magenta) on HylA (PDB: 8FYG [www.rcsb.org/structure/unreleased/8FYG]) crystal structure that were mutated to corresponding HylB residues. The HA-6 ligand is taken from the SpnHyl crystal structure (PDB: 1LOH). B-f, HPLC profile of HMW-HA after 24 hr coincubation with WT or mutant HylA (0.35 μg): HA alone (b), rHylA (c), or rHylA with single amino acid substitutions (d-f). g, HPLC profile of HMW-HA after 24 hr coincubation with WT rHylB (0.35 μg). h, quantification of HA-digested peaks was performed using known concentrations of purified HA oligosaccharides. I, water alone run as a blank control. Asterisk (*) in b-i represents non-specific peaks, present in water control as well. Data are representative of two independent experiments.

FIG. 21 (panels a-h) shows the proinflammatory properties and TLR2 dependence of Hyl degradation products. A-c, HaCaT cells were stimulated with HA, that had been predigested with either rHylA or rHylB, for 24 hr followed by IL-6 (a), IL-8 (b) and TNF-α (c) measurements in the culture supernatant. D, HaCaT cells were stimulated with the HA, predigested with either supernatant from HL043PA1, HL110PA3 or corresponding isogenic mutant for 24 hr followed by IL-6 measurement in the culture supernatant. E-g, WT, TLR2^{-/-} and TLR4^{-/-} mice were infected i.d. with WT or isogenic ΔhylA HL043PA1 (2×10⁷ CFU) strain as above. Disease score I and skin cytokines (f, g) at 24 hr post-infection. H, IL-6 in WT or TLR2^{-/-} BMDM culture supernatant after stimulation with rHylA or rHylB digested HA. Data in a (n=5 for HA+rHylA and 6 for other conditions), b (n=3 for media and 6 for other conditions), c (n=6), d (n=10), h (n=4), are presented as mean±SD and each data point represents one well. The data are representative of two independent experiments. E-g, Bars denote median, and each data point represents one individual mouse (n=5 TLR4^{-/-}, n=8 for TLR2^{-/-} or n=11 for WT mice infected with HL043PA1, and n=4 for TLR4^{-/-}, n=5 for TLR2^{-/-} or n=6 for WT mice infected with isogenic ΔhylA). The p values in a, b were calculated by one-way Welch ANOVA test, p values in c, d were calculated by non-parametric Kruskal-Wallis one-way ANOVA test, and p values in e-h were calculated by non-parametric two-tailed Mann-Whitney U test.

FIG. 22 (panels a-f) shows selective neutralization of HylA improves acne lesions and mitigates inflammation. A,b, CD1 mice (n=10) immunized with either Alum (Mock) or Alum-rHylA (HylA) were challenged i.d. with HL043PA1. Disease score (a) and IL-1b in skin homogenate at d2 post-challenge. C-e, mice (n=15) were immunized intraperitoneally (i.p.) with alum plus C-terminus of tetanus protein (TT) or multiple HylA epitopes linked to TT (mEHylA), then challenged i.d. with HL043PA1 C. acnes strain. Disease score (c) bacterial burden (d), and IL-1b I at d2 post-challenge. F, serum (1:100,000 diluted) anti-HylA or anti-HylB IgG antibody titers after the third immunization with mEHylA vaccine. Bars denote median. Data are from two (a, b, f) or three (c-e) independent experiments with each data point representing one mouse. The data in a-e were analyzed by non-parametric two-tailed Mann-Whitney U test, and in f by non-parametric Kruskal-Wallis one-way ANOVA test.

FIG. 23 (panels a-c) shows the HylB digest analyzed by HPLC and LC-MS demonstrates only HA-disaccharide. A, HPLC of HylB digest (50 ug HA+1 ug HylB for 24 hr) demonstrates 2 peaks, 12.12 and 10.48, which correspond to the isomeric forms of HA-disaccharide. B, LC-MS showing that the peak from (a) is HA-disaccharide by m/z. c, HPLC shows no evidence of HA-tetra-saccharide from HylB digest.

FIG. 24 (panes a-e) show crystal structures of HylA and HylB, and functional regions of Hyl from C. acnes. A-c, HylA and HylB (wild-type and mutant) crystal structures are shown. D, superimposition of the three crystal structures of HylA (PDB: 8FYG [www.rcsb.org/structure/unreleased/8FYG]) and HylB (PDB: (wild-type 8FNX [www.rcsb.org/structure/unreleased/8FNX]) and mutant (PDB: 8G0O [www.rcsb.org/structure/unreleased/8G0O])) crystal structures are shown. D, superimposition of the three crystal structures of HylA and HylB are shown superimposed. E, the putative functional parts of the Hyl enzyme from C. acnes are shown in the electrostatic surface view (prepared by APBS Electrostatics, PyMOL Molecular Graphics System, Version 2.4 Schrödinger, LLC.) of the HylA. All these regions, including positively charged cleft area, aromatic/hydrophobic patch, active center, and negative patch, are located around the substrate-binding cleft. It also depicts the substrate entry and products release ports at the cleft region. These structural regions in the homologous enzymes, including ScHyl (PDB: 2X03), SpnHyl (PDB: 2BRW), and SaHyl (PDB: 1FIS), were shown to be involved in substrate attraction, binding, positioning and translocation, and product release.

FIG. 25 (panels a-b) show HA degradation product from HA incubation with mutant rHylA proteins. A-b, HPLC profiles of HMW HA (2 mg/ml plus 0.35 μg recombinant protein) digested for 24 hr with mutant N442D rHylA (a) or E346G (b). Asterisk (*) in a, b represents non-specific peaks, present in water control as well. Green circle (a) shows larger oligomers. Data are representative of two independent experiments.

FIG. 26 shows domain motions in HylA-wt, HylB-wt and mutants of HylA. Normalized amplitudes of the four types of domain motions in each model are shown. The cleft opening/closing motion (Evec1), domain twisting motion (Evec2), substrate entry opening/closing motion (Evec3), and product-exit opening/closing motion (Evec4) were calculated from the molecular dynamics simulations by GROMACS version 2022.4.

FIG. 27 (panels a-k) show the effect of mEHylA vaccination on disease induced by HL043PA1 or HL110PA3 infection. A-b, mice (n=15) were vaccinated, as in FIG. 17, with either mock (Alum-TT) or Alum plus mEHylA (mEHylA) and challenged 14d post-last vaccination with HL043PA1. TNF-α (a) and IL-6 (b) at d2 post-infection. C-f, mice (n=5) were vaccinated, as above with either mock (Alum-TT) or Alum plus mEHylA (mEHylA) and challenged 14d post-last vaccination with HL110PA3. Disease score (c), IL-1B (d), IL-6 I, and TNF-α (f) at d2 post-infection. G-k, CD1 mice (n=5) were vaccinated as above with either mock (Alum-TT) or Alum plus mEHylA (mEHylA), and then 10d post-last vaccination total CD3+T cells were isolated from spleen and adoptive transferred into naïve recipient mice, followed by HL043PA1 challenge 20h later. CFUs (g), disease score (h) and cytokines (i-k) were measured on d2 post-infection. Bars denote median. Data are from three independent (a-b) or from one experiment (c-k), with each data point representing one mouse. The p values in a-f were calculated by non-parametric two-tailed Mann-Whitney U test and by one-way ANOVA for g-k.

FIG. 28 (panels a-f) shows HylA enzyme neutralization assay. A-e, HPLC profile of HMW-HA (2 mg/ml) after 20 hr coincubation with rHylA (0.3 μg) and serum (10 μl): HA standard (a), HA alone (b), water ran as blank (c), Mock serum (Alum-TT)+rHylA+HA (d), and anti-mEHylA serum+rHylA+HA I. Asterisk (*) in a-e represents non-specific peaks, present in water control as well. Pooled serum (n=5) was used for the assay and performed in duplicate for mock serum and in triplicate for anti-mEHylA serum. F) Antibody isotype titers (serum diluted 1:100,000) in sera isolated from mice vaccinated with either mock (Alum-TT) or Alum plus mEHylA (mEHylA) at d14 post-last vaccination. Statistical analysis in f was performed by one-way Anova with Tukey's post-hoc test. Green circle represents larger oligomers.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared.

“Linked” as used herein in the context of linked peptides, polypeptides, or proteins refers to being “connected to” either directly or indirectly. Indirect linkage can be mediated by a polypeptide linker such as poly-glycine or a glycine-serine polypeptide, for example, (GGGGS)n (SEQ ID NO:5)n or (GGGGGS)n (SEQ ID NO:6)n, wherein n is an integer. In various embodiments, n is an integer from 1-10, 10-20, 20-30, 30-40, or 50-100. Other such linkers are known in the art and is considered to be encompassed by this term.

Linear epitope score can be determined by a semi-empirical method which makes use of physicochemical properties of amino acid residues and their frequencies of occurrence in experimentally known segmental epitopes to predict antigenic determinants on proteins. The method is based on a single parameter that predicts the antigenic score based on the antigenic propensity as described by Kolaskar and Tongaonkar (1990). A. S. Kolaskar and Prasad C. Tongaonkar. FEBS. 276 (12). 172-174 (1990).

Herein, we showed that HylA hydrolyze HA into large-sized HA fragments that drive robust TLR2-dependent inflammatory pathology. In contrast, HylB degrades hyaluronic acid (HA) exclusively to HA disaccharides leading to reduced acne immunopathology. Structural and phylogenic studies suggest that the enzymes evolved from a common hyaluronidase that acquired diverging enzymatic properties. We showed that selective inhibition of HylA by vaccination alleviates acne pathology, thus pointing to a virulence-based approach to acne treatment. We recently addressed growth of C. acnes in mice by applying human synthetic sebum to murine skin with infection that permitted persistence of C. acnes. We further demonstrated remarkable and uniformly enhanced pathogenicity of acne-associated strains compared to health-associated strains in the model. Using this model, we set to address the question—what genetic element(s) drove the divergence of C. acnes disease or health association.

Among the candidate factors revealed by comparative genomic studies of health-versus acne-associated strains, we were particularly intrigued by a matrix degrading enzyme hyaluronidase (Hyl) that in mammals generates HA fragments mediating inflammation via TLR2/4 signaling, which is a major proinflammatory pathway in acne pathogenesis. Two variants of the Hyl enzyme, HylA and HylB, are expressed by C. acnes, which appear to be distinctly expressed by acne- and health-associated strains, respectively. We therefore sought to gain a greater understanding of their relationship and their contribution to C. acnes health and acne association.

Our study supports Hyl as a major virulence factor that explains the divergence of health and acne phenotype of C. acnes strains. This is supported by the high degree of association between HylA and HylB with clinical disease or health, their TLR2 dependency in immunopathologic mechanisms, consistent with acne vulgaris, and the contribution of the two Hyl variants to immunopathology and health. Although our data support the prominence of HylA virulence, several other C. acnes virulence factors have been reported. These include toxic porphyrin biosynthesis genes that are upregulated with vitamin B12 supplementation, and CAMP factor which enlist cytotoxic host sphingomyelinase.

Based on our phylogenetic analysis, HylA is the only proinflammatory Hyl elaborated by a human commensal or pathogen. Since C. acnes is both a human commensal and a soil bacterium, its clustering among environmental microbes in the phylogeny tree makes sense, perhaps as a transition from soil to commensal. HylA and HylB relatedness to Hyl from soil derived organisms, Streptomyces and Arthrobacter, can be consistent with this proposed transition from a multi-functioning lyase to a more restrictive and processive enzyme. Because an inflammatory milieu is usually harmful to pathogens, the expectation is that C. acnes Hyl would evolve from pro-inflammatory HylA to HylB. While this was anticipated, expression of HylA or HylB do not appear to modify C. acnes survival in our acne model to exert a selective pressure on survival. This would be consistent with the finding of abundant C. acnes strains that express either of the Hyl. Although HylA and HylB differ by 26% in genomic sequence, we show that one single amino acid substitution can significantly alter the phenotype of the enzyme, suggesting that a major pathogenic potential of C. acnes may only be encoded and modified by small changes that occur during the evolution of the enzyme. These single amino acid substitutions are observed to be conserved across the different C. acnes strains.

It has been reported that health- and acne-associated C. acnes acquired HylB and HylA, respectively, through different insertional events in the indel 14 genomic region, based on finding of different sequences up and downstream of hylA and hylB. It is unclear how these events occurred, but the structure and sequence relatedness of HylA and HylB compared to other bacterial Hyls suggest that they most likely originated from within Cutibacterium species. Until further genetic data become available, current data are most consistent with that interpretation.

The understanding of the structural differences between HylA and HylB allowed us to devise selective therapeutics that target the proinflammatory enzyme. A report showed that only 4-17% of humans develop neutralizing antibodies to C. acnes Hyls and only after early adulthood. Hence, a vaccine approach would be of significant benefit.

C. acnes Hyaluronidases Contribute to Healthy or Acneic Skin

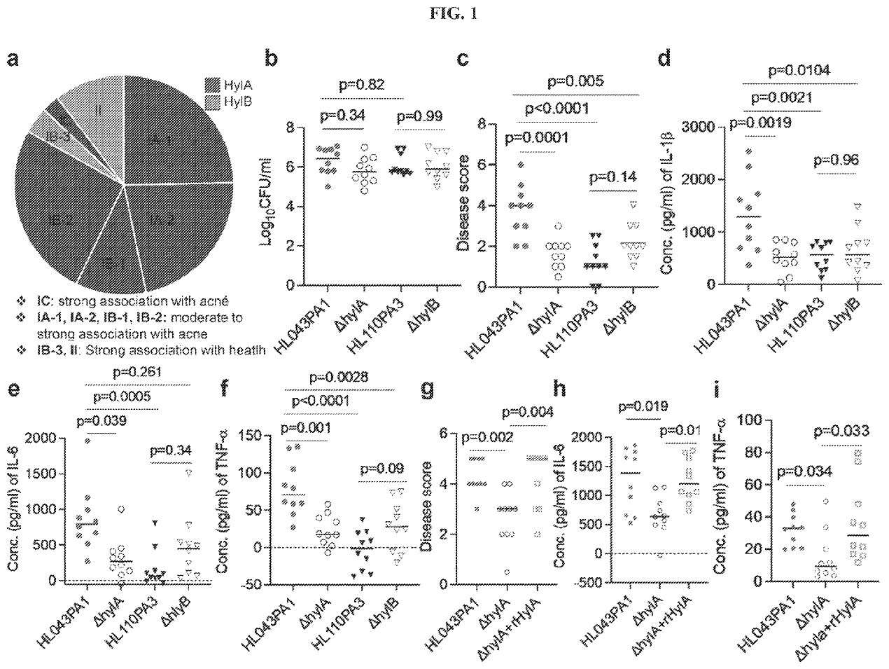

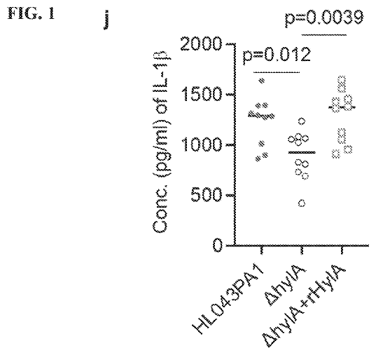

To assess the potential importance of HylA or HylB enzyme in clinical acne, we surveyed all hylA and hylB genes in the NCBI and profiled their association with health- and acne-associated C. acnes strains (Table 1). C. acnes strains are classified into phylotypes, which have different predilection for association with acne. Phylotypes IA-1, IA-2, IB-1, IB-2 and IC are associated with acne, whereas phylotypes IB-3 and II are closely associated with health (FIG. 1a). A different classification based on the presence of ribotype (RT) demonstrates strong association of RT2/6 with health and RT4/5 with acne disease (Table 1). As shown in FIG. 1a, hylA gene is found almost exclusively in acne-associated phylotypes and hylB in health-associated phylotypes, supporting their potential to contribute to acne or health.

TABLE 1
Table showing C. acnes phylotypes, number and percentage
of strains in each phylotype, the presence of Hyl (A or
B) gene and association with acne or healthy skin.

| | Number | % of total | | Health/acne | |
|---|---|---|---|---|---|
| Phylotypes | of strains | strains | Hyl | association | Ribotype |
| IA1 | 19 | 14.63 | A | Acne | 5 |
| IA2 | 17 | 13.09 | A | Acne | 4/5 |
| IB1 | 8 | 6.16 | A | Acne | 3/8 |
| IB2 | 20 | 15.4 | A | Acne/health | 3/16 |
| IB3 | 3 | 2.31 | B | Healthy skin | 2/1 |
| IC | 2 | 1.54 | A | Acne | 5 |
| II | 8 | 6.16 | B | Healthy skin | 1/2/6 |

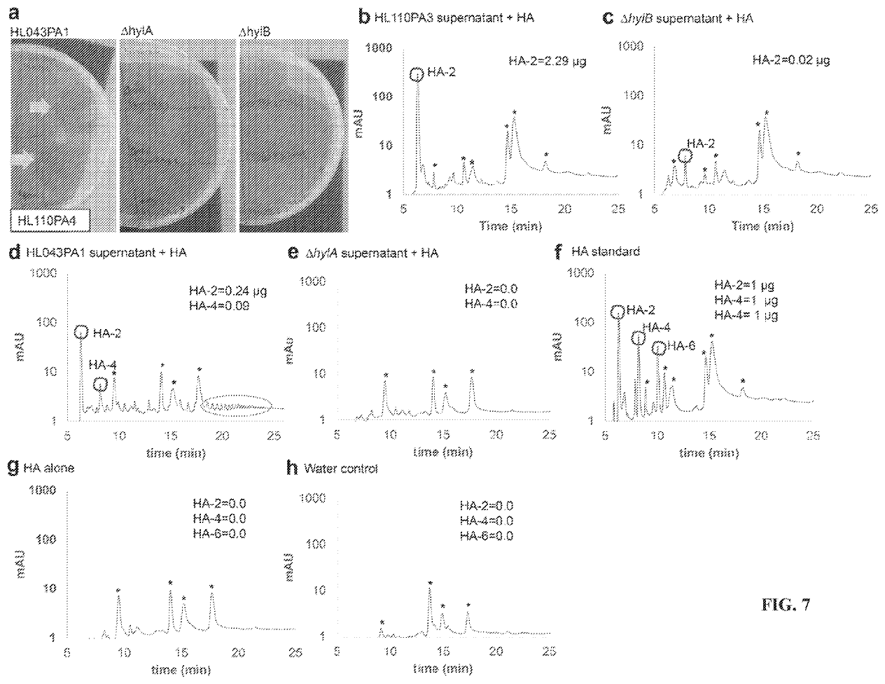

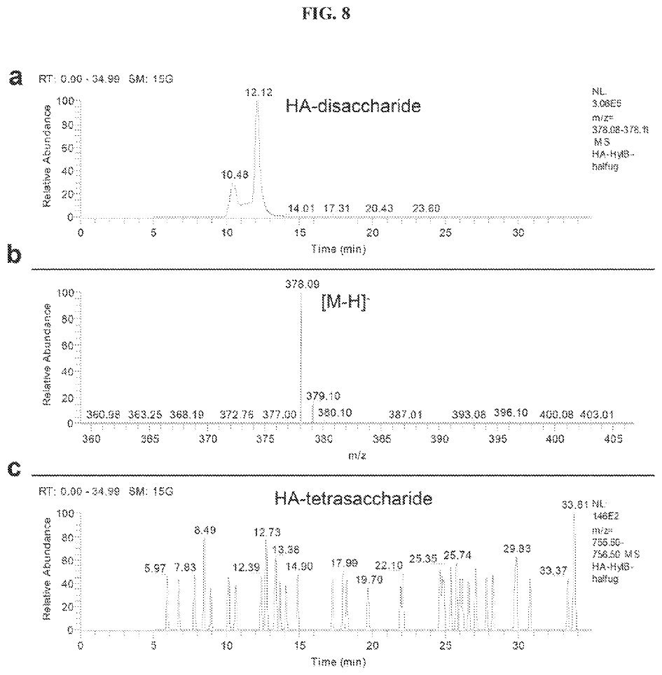

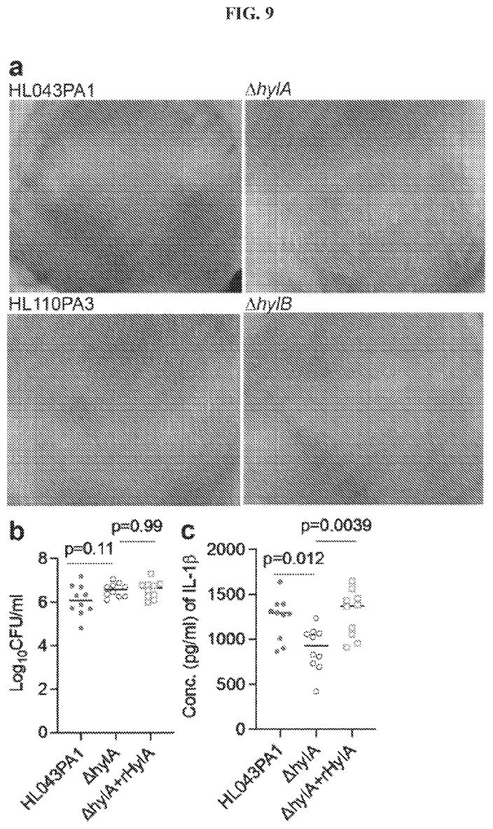

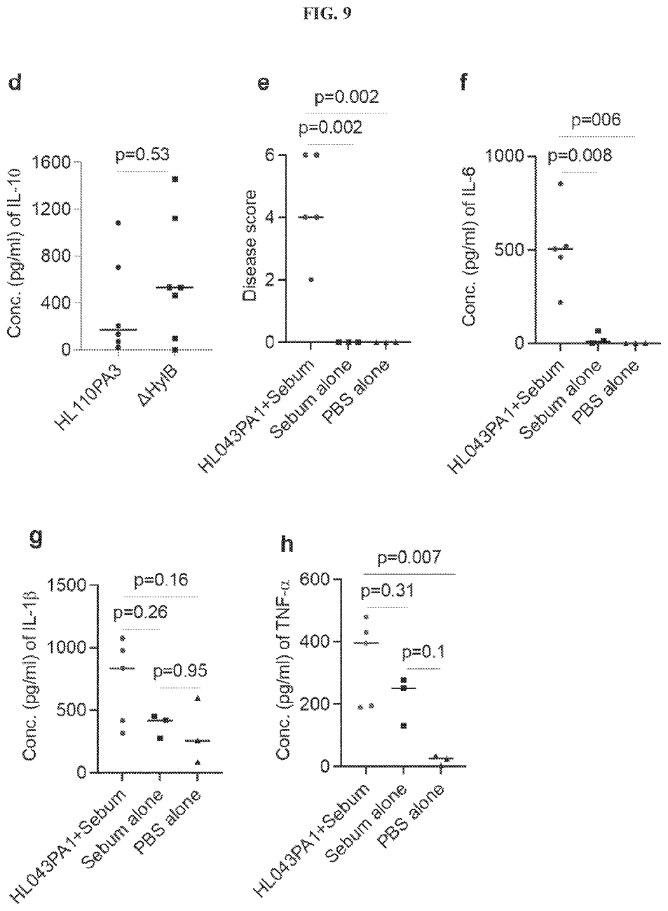

To directly query the role of hylA and hylB in acne, we generated in framed allelic exchange of hylB and hylA in prototype health (HL110PA3, Phylotype II, RT6) and acne (H043PA1, Phylotype IA-2, RT5) strains, respectively. From our prior study, these two strains represented the least and most acnegenic strains of the panel of RT2/6 and RT4/5 health and acne strains tested in our acne mice model, respectively. We verified Hyl gene deletion by sequencing and loss of Hyl activity by HA plate assay and HPLC (FIG. 7a-h and FIG. 8). We applied both WT and mutant strains to our murine acne model, and surveyed disease score and tissue cytokines after 2 days (FIG. 9a). Independent of bacterial burden (FIG. 1b), the hylA deletion mutant induced dramatic reduction in disease score and proinflammatory cytokines, compared to the parent acne-associated strain (FIG. 1c-f). Conversely, the hylB deletion mutant demonstrates a modest increase in disease score and pro-inflammatory cytokines compared to the health-associated parental strain (FIG. 1c-f), consistent with the interpretation that HylB has anti-inflammatory properties. Notably, when comparing the immunopathology of acne induced by the prototype acne- and health-associated C. acnes strains, the difference in pathology between the acne and health strains is abrogated or even modestly reversed in the absence of Hyl enzymes, pointing to the important contribution of the Hyl variants to differences between the health and acne strains.

We provided corroboration of HylA proinflammatory phenotype by complementation of ΔhylA with WT HylA recombinant protein (rHylA) injection (FIG. 1g-i; FIG. 9b,c). Overall, our findings suggest that the two Hyl variants play a major role in promotion or modulation of acne immunopathology with HylA.

HylA and HylB Enzymes have Distinct HA Degradation Pattern and Efficiency

We next asked how HylA/B developed such distinct inflammatory properties? Reports have shown that mammalian and Streptomyces hyalurolyticus enzyme produce HA fragments larger than 4mers that contribute to the induction of pro-inflammatory cytokines. More recently, we showed that pathogen bacterial pathogens (Group B Streptococcus, S. pneumoniae and S. aureus) generate Hyls that degrade proinflammatory HA strictly to non- or anti-inflammatory disaccharides (HA-2). Hence, enzymatic activity of HylA and HylB can lead to different inflammatory outcome depending on the HA degradation product.

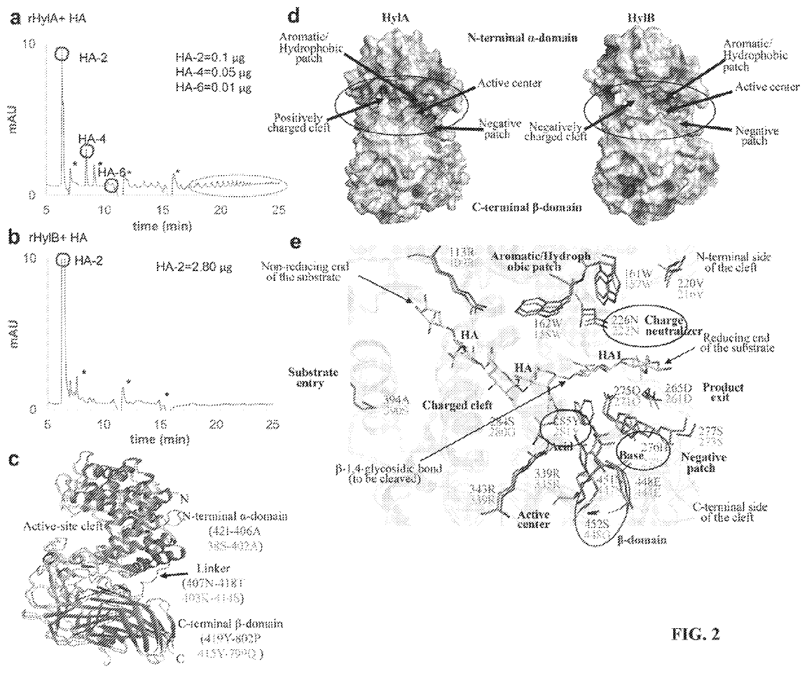

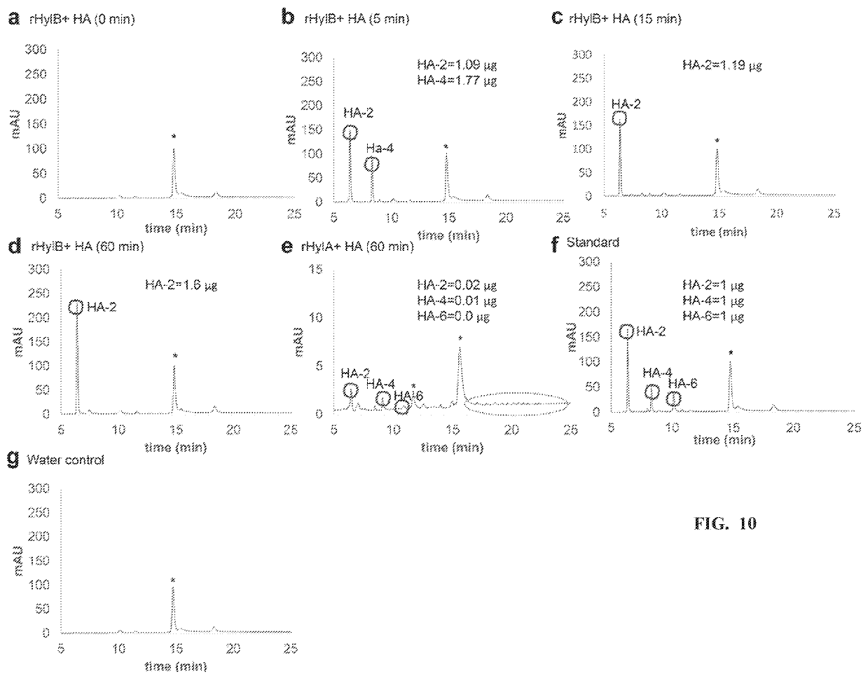

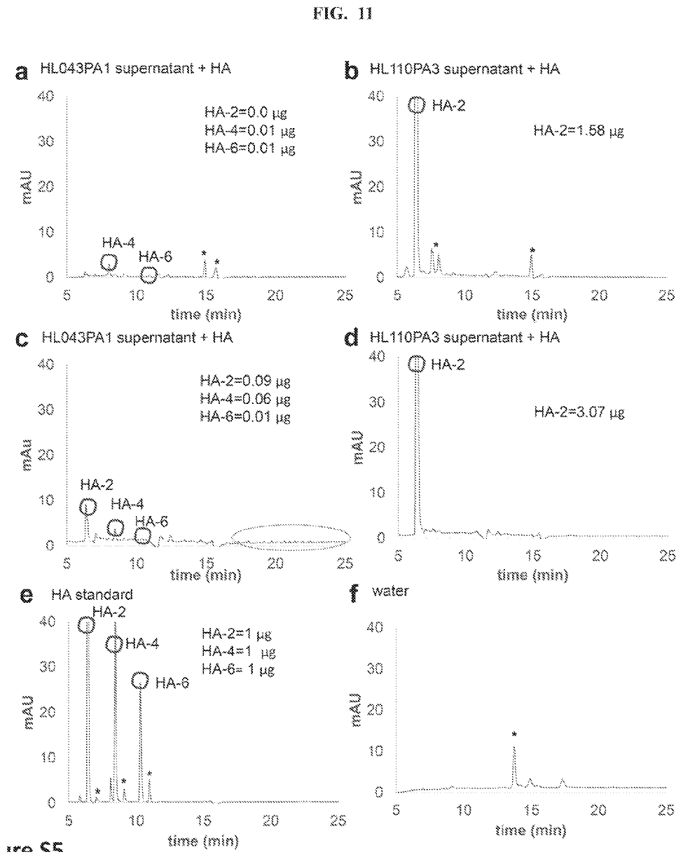

Hence, we incubated supernatant from HL110PA3 or H043PA1, or recombinant HylA or HylB with high molecular weight (HM) HA for 1 or 24h (FIG. 10a-e and FIG. 11a-d). HylB enzymatic activity is rapid and produces predominantly HA-2 from the start through the end of the reaction (FIG. 2a). Tetrasaccharides HA (HA-4) is briefly observed at 5 minutes (FIG. 10b), but thereafter, only HA-2 is detected as degradation reaction continued.

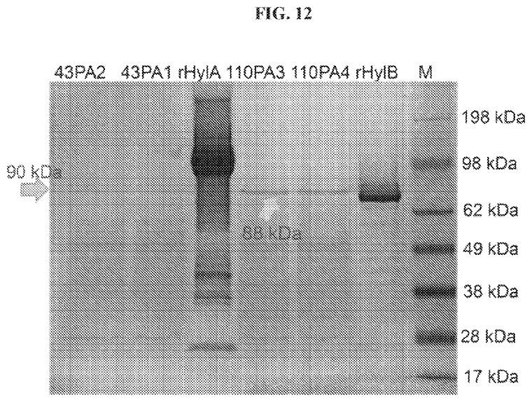

In comparison, HylA produced different oligosaccharides throughout the reaction course, including HA-4 and hexasaccharides HA (HA-6), along with the HA-2 (FIG. 2b). Notably, at the end of 24h, HA-4 and H-6 and higher MW HA persist. We have performed exhaustive digestion with HylA-containing supernatant for up to 6 days and demonstrated that digestion is still incomplete These differences in the degradation rate and pattern are consistent with findings from another group on 3 HylA (phylotype IA) and 2 HylB (phylotype IB and II) producing strains. They suggest a fundamental difference in degradation mechanisms—a processive/progressive exolytic degradation mechanism for HylB and a combination of this and ‘no-processive random bite endolytic’ degradation mechanism by the HylA. Although lower amounts of HylA compared to HylB is secreted into supernatants (FIG. 12), the difference in endolytic versus processive mechanism were not overcome by increased enzyme concentration (FIG. 10c-e and FIG. 11a,b).

### HylA and HylB Functional Divergence Promotes Distinct Mechanisms of HA Degradation

Unlike human Hyls, enzymes secreted by commensal or pathogenic bacteria reported to date, degrade HA strictly to HA-2. A single cluster of bacteria stands out in the Hyl phylogenetic tree for ability to generate fragments larger than disaccharides. This cluster includes environmental bacteria, such as Streptomyces that degrade HA into large fragments. Cutibacteria, which are both a human commensal and environmental bacteria, also cluster alongside Streptomyces, thus raising the question if proinflammatory HylA derives from Streptomyces and anti-inflammatory HylB from bacterial pathogens such as Streptococci. HylA and HylB are ninety percent homologous in nucleotide sequence and 74% identical in amino acid sequence. Further investigation of HylA/B homologous enzymes across related species show that the two enzymes most likely originated from within Cutibacterium lineage.

Structures of HylA and HylB Reveal High Structural Similarity Between them and with Homologous Glycosaminoglycan-Degrading Lyases from Other Bacteria

To understand the structural basis for the differences in the hyaluronate lyase activities of HylA and HylB, we solved the X-ray crystal structures of HylA Y285F mutant and wild-type (WT) HylB to 2.05 Å and 2.1 Å, respectively.

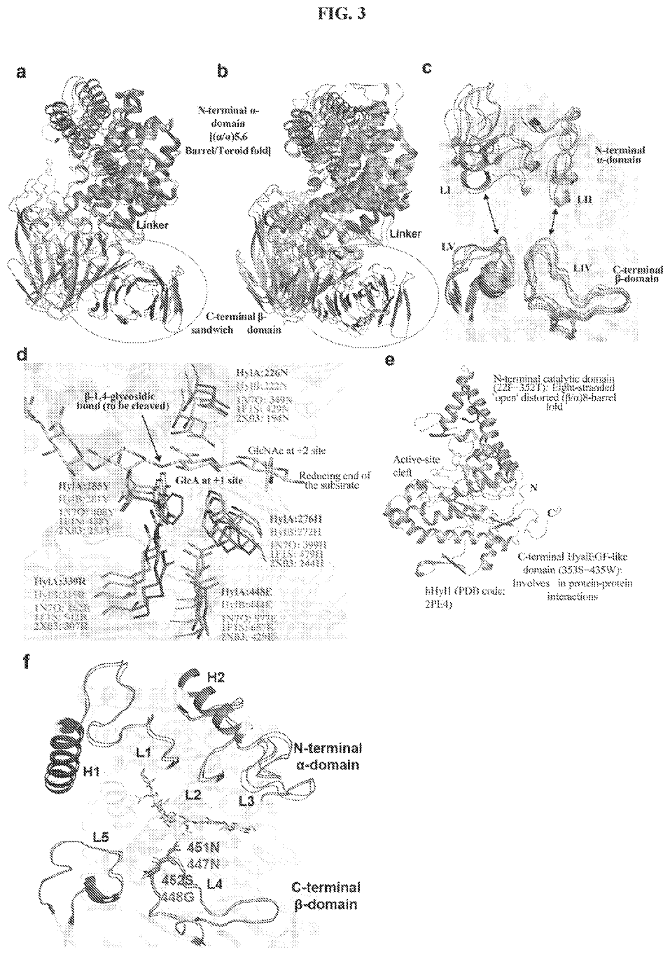

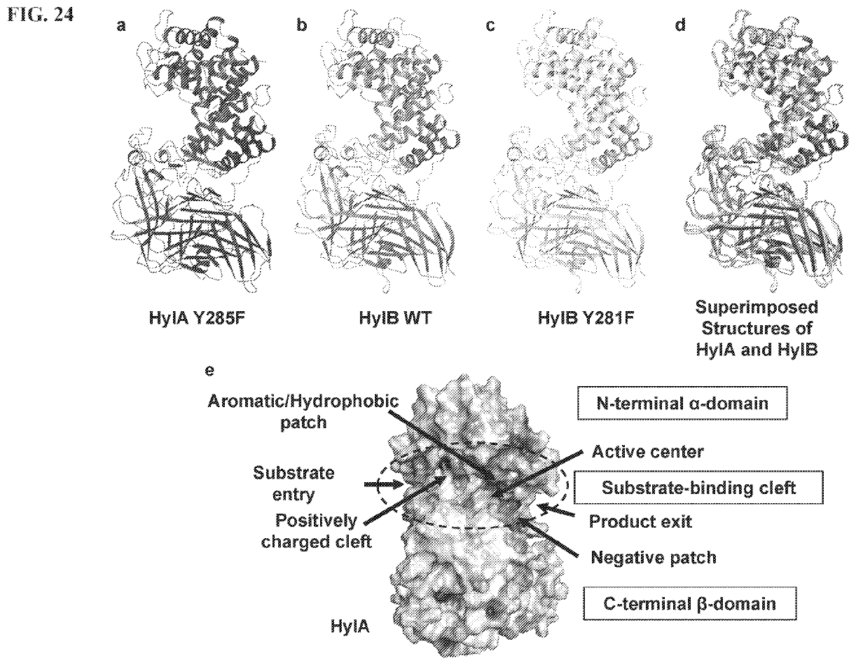

HylA Y285F is a catalytically deficient form of the enzyme, and the structure is hereafter referred to as “HylA.” Befitting enzymes with 74% identity between them, the structures are highly similar, overlaying with a r.m.s.d. of 0.8 Å over 751 residues (both HylA molecules in the crystallographic asymmetric unit vs. HylB). Typical of hyaluronate lyases, HylA and HylB consist of a mostly a-helical N-terminal domain, a C-terminal domain comprising mainly of β-strands, and a catalytic site in a large cleft predominantly within the N-domain (FIG. 2c, d). The catalytic sites overlay closely, containing many elements conserved in hyaluronate lyases, including two conserved tryptophans (HylA/B Trp161/157 and Trp162/158), several positively charged residues, and the three residues of the catalytic triad (Asn226/222, His276/272, and Tyr285/281) (FIG. 2e). We further solved the structure of the catalytically deficient Y281F mutant of HylB to a resolution of 2.1 Å (Table 2); this structure was solved in a different space group than wild-type HylB (P1 with two molecules in the crystallographic asymmetric unit vs. P41212 and one molecule for wild-type). Though crystallized with different crystal-packing interactions, HylB Y281F is nearly identical in conformation to wild-type HylB (r.m.s.d 0.6 Å, wild-type vs. both Y281F molecules) (FIG. 24a-e). We were unable to produce crystals of HylA or HylB complexed with HA fragments; indeed, the three structures reported here display open catalytic clefts likely incompatible with binding the substrate (FIG. 3).

TABLE 2

| Parameter | HylB WT | HylB Y281F | HyIA Y285F |
|---|---|---|---|
| PDB code | 8FNX | 8G0O | 8FYG |

Data collection

| | | | |
|---|---|---|---|
| Space group | P 41 21 2 | P 1 | P 1 |

Unit cell constants

| a, b, c (Å) | 103.68,103.68, 172.94 | | 52.98, 53.01, 161.02 | | 51.96, 59.42, 125.99 | |
|---|---|---|---|---|---|---|
| α, β, γ (°) | 90.00, 90.00, 90.00 | | 91.95, 98.03, 114.56 | | 90.61, 95.74, 90.08 | |
| Resolution range (Å) | 19.73-2.1 | (2.21-2.1)^{#} | 19.72-2.1 | (2.21-2.1) | 19.8-2.05 | (2.1-2.05) |
| Data completeness (%) | 99.70 | (97.8) | 92.6 | (87.7) | 93.9 | (90.59) |
| Total reflections | 784293 | (102767) | 161839 | (21056) | 200582 | (16051) |
| Unique reflections | 55667 | (7843) | 85175 | (11885) | 88266 | (6191) |
| Multiplicity | 14.1 | (13.1) | 1.9 | (1.8) | 2.2 | (1.9) |
| Mean I/sigma(I) | 14.50 | (4.3) | 5.4 | (1.7) | 7.33 | (1.99) |
| Wilson B-factor (Å²) | 18.04 | | 15.3 | | 23.30 | |
| R-merge | 0.172 | (0.672) | 0.12 | (0.548) | 0.097 | (0.497) |
| R-meas | 0.178 | (0.699) | 0.17 | (0.775) | 0.128 | (0.662) |
| CC1/2 | 0.997 | (0.892) | 0.981 | (0.853) | 0.991 | (0.623) |
| CC* | 0.999 | (0.967) | 0.995 | (0.894) | 0.998 | (0.893) |

Refinement

| | | | | | | |
|---|---|---|---|---|---|---|
| Reflections used in | 55667 | (7843) | 85104 | (8160) | 88252 | (5832) |
| refinement | | | | | | |
| Reflections used for R-free | 2000 | (283) | 2061 | (235) | 2006 | (138) |
| R-work | 0.1564 | (0.2034) | 0.2525 | (0.339) | 0.2252 | (0.319) |
| R-free | 0.1930 | (0.2527) | 0.2685 | (0.355) | 0.2579 | (0.35) |
| CC(work) | 0.969 | (0.914) | 0.893 | (0.674) | 0.931 | (0.713) |
| CC(free) | 0.955 | (0.820) | 0.875 | (0.595) | 0.904 | (0.681) |

No. of non-hydrogen atoms

| | | | |
|---|---|---|---|
| Total | 6842 | 12210 | 12845 |
| Macromolecules | 5830 | 11540 | 11485 |
| Ligands | 52 | 0 | 28 |
| Solvent | 960 | 670 | 1332 |
| Protein residues | 762 | 1519 | 1522 |
| Rotamer outliers (%) | 0.49 | 1.58 | 0.50 |
| Clashscore | 2.57 | 6.40 | 6.36 |

Average B-factor (Å²)

| | | | |
|---|---|---|---|
| Overall | 20.96 | 21.64 | 26.90 |
| Macromolecules | 19.29 | 21.69 | 26.37 |
| Ligands | 36.49 | N/A | 27.62 |
| Solvent | 30.23 | 20.80 | 32.04 |

R.m.s. deviations

| | | | |
|---|---|---|---|
| Bond lengths (Å) | 0.007 | 0.003 | 0.002 |
| Bond angles (°) | 0.83 | 0.60 | 0.55 |

The crystal structures show that C. acnes HylA and HylB share high structural similarity with glycosaminoglycan lyases from gram-positive bacteria, including hyaluronate lyases from Streptococcus agalactiae and Streptococcus pneumoniae, xanthan lyases from Bacillus sp. Strain GL1 and Paenibacillus nanensis, and chondroitin AC lyases from Streptomyces coelicolor and Arthrobacter aurescens (FIG. 3a-c). Each of these enzymes is homologous to HylA and HylB, with sequence identities ranging from 23-37% vs. HylA, and are structurally homologous as well, with root mean square deviations vs. HylA ranging from 2.2-3.4 Å. Additionally, the geometry of catalytic residues conserved within these enzymes is maintained in HylA and HylB (FIG. 3d, e).

To obtain further insight into the functional divergence of HylA and HylB, we identified four residue positions in the catalytic cleft that differ between HylA and HylB. Two of these residue pairs (HylA Arg397/HylB Val393 and HylA Ser116/HylB Glu112) are located deep in the cleft in proximity to the β-D-glucuronic acid moiety at the non-reducing end of the putative bound HA, and contribute to the binding cleft of HylA displaying a more positively-charged surface than that of HylB (FIG. 2d). The other two pairs (HylA Asp345/HylB Asn341 and HylA Glu346/HylB Gly342) lie closer to the predicted position of the preceding β-D-glucuronic acid moiety.

Next, we examined these residue pairs by mutating the HylA residues to match their HylB counterparts and vice versa, and measured their hyaluronidase activity to determine which amino acid of each pair is favored at that position. In this assay, the cleavage of HMW-HA by hyaluronidase is recorded by monitoring UV absorbance at 232 nm, which increases with the formation of an unsaturated carbon-carbon bond in the β-D-glucuronic acid moiety at the cleavage site.

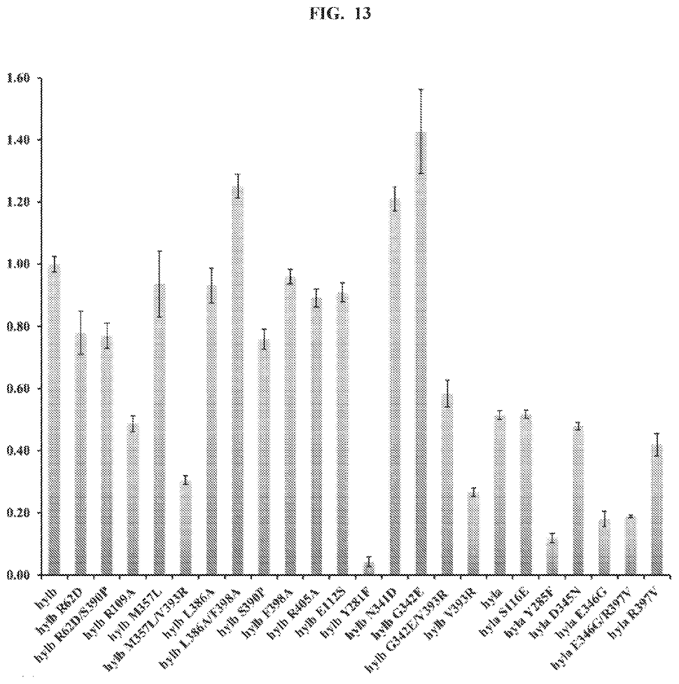

This assay shows that HylB degrades HA at approximately twice the rate of HylA, while control mutations of the tryptophan residues of the catalytic triad (HylA/B Y285F/Y281F) severely curtail the HA-degrading activity of both enzymes (FIG. 13). For the residue pairs, only HylA/HylB position 346/342 showed a distinct preference, with both HylA and HylB displaying greater enzyme velocity with glutamic acid over glycine. Incongruously, however, the wild-type sequence that contains glutamic acid at this position is not that of HylB but HylA, the less active of the two variants; it is thus unlikely that this residue accounts for part of the difference in cleavage rate between HylA and HylB.

### Mutation of HylA Residue Ser 452 to Glycine of HylB Alters HylA Enzymatic Phenotype

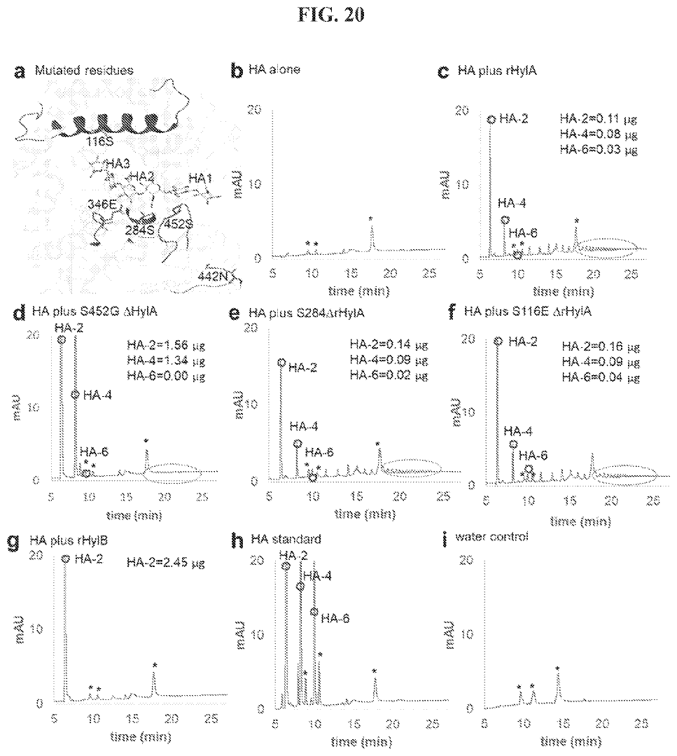

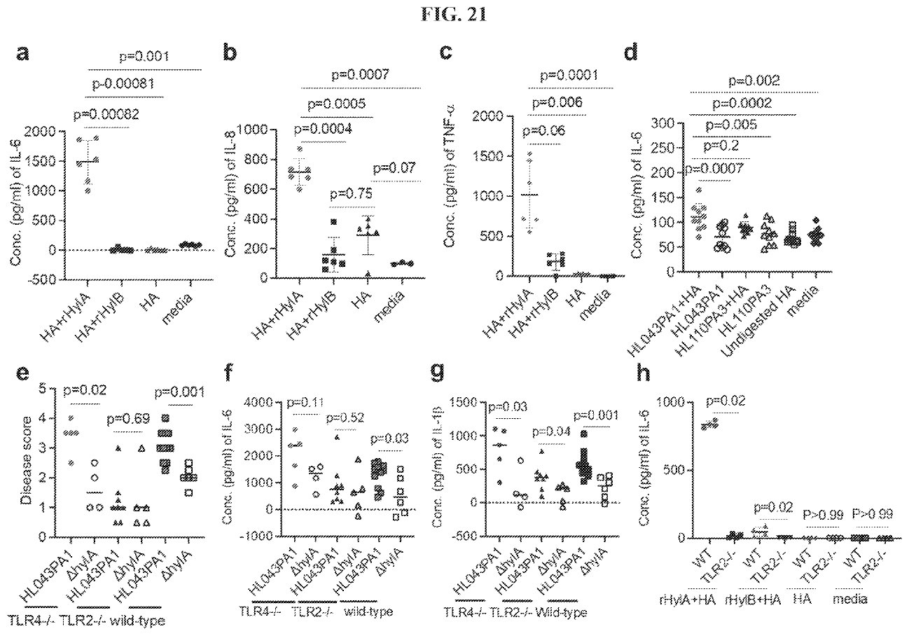

We further generated point mutations in several residues of HylA to the analogous residues in HylB to recapitulate the HylB phenotype of HA product size. HylA S452G is located in a loop in the C-terminal domain, between strands β10 and β11 (FIG. 3d, FIG. 20a); mutation of the preceding residue in the analogous loop of S. pneumoniae Hyl has been shown to alter enzymatic activity. HylA S116E and E346G, as noted above, reside in the catalytic cleft towards the non-reducing end of the HA substrate. HylA S284G lies adjacent to the catalytic Y285, and N442D is placed at an exposed position within the C-terminal domain (FIG. 20).

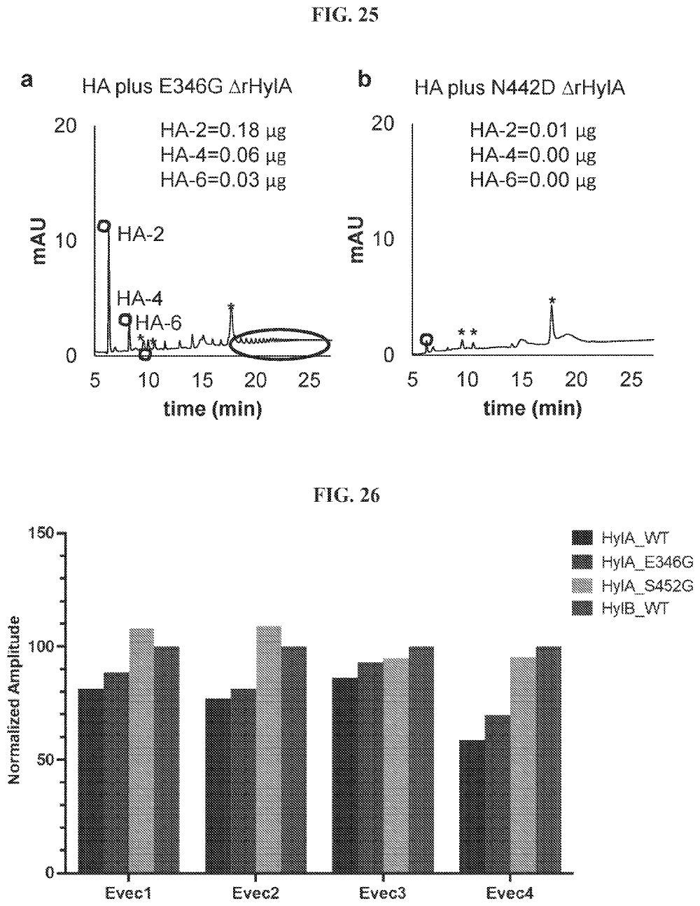

HylA S284G, S116E, and E346G had no significant effects on the HylA product size phenotype, though E346G showed decreased overall enzymatic activity and N442D resulted in a nearly complete loss of activity (FIG. 20-i, and FIG. 13 and FIG. 25). HylA S452G, however, successfully altered the HylA enzymatic phenotype; reminiscent of HylB, S452G displayed an increased enzyme velocity and reduced amounts of larger oligomers as product (FIG. 20). The resulting product size, however, was not predominantly HA-2 as would be expected for a strictly HylB-like phenotype, but was a mixture containing a higher ratio of HA-4 to HA-2 compared to WT HylA. Interestingly, the amino acid residue, S452 in HylA and G448 in HylB, is conserved across C. acnes strains suggesting that a similar hydrolytic process may be conserved.

Earlier domain motions in SpHyl, SaHyl and ScHyl enzymes have been implicated in substrate processing by molecular simulation. To understand whether substrate processing by HylA and HylB follow a similar mechanism, we performed molecule simulation of HylA and HylB wild-types and HylA-mutants, S452G and E346G as described by Josh et al. Our molecular simulation study results are consistent with observations of Josh et al.; Briefly, PCA analyses of simulated trajectories suggest that HylB-WT is far more dynamic than HylA-WT and S452G mutation in HylA shows increased (amplitude) domain motions similar to HylB-WT; the cleft opening/closing motion (Eigenvector 1) increased by about 20%, while the other domain motions increased by 10-40% (FIG. 26. These observations are consistent with the hypothesis that complex structural dynamics are one of the key mechanisms for substrate processing by HylA and HylB.

Additionally, the overall three-dimensional structures of HylA and HylB are almost identical (FIG. 2c); the fold consists of an N-terminal α- and a C-terminal β-domains connected by 12-residue long linker. The substrate binding cleft in both the enzymes contain highly a conserved catalytic site (FIG. 2d and Table 3) and is decorated with charged residues. Most differences are observed in the vicinity of the substrate binding region (Table 4). Comparing the substrate binding domains to the other Hyls from Streptomyces and Streptococcus species suggests that a positive patch in HylA/B is located towards the non-reducing end of the substrate and a negative patch is located at the reducing end, are involved in substrate binding and product release, respectively, with few aromatic residues forming an aromatic/hydrophobic patch implicated to be involved in substrate positioning for catalysis (FIG. 2e and Table 3).

TABLE 3
Active site cleft components at residue level

| Active site cleft | Domain | | |
|---|---|---|---|
| component | contribution | HylA | HylB |
| Charged cleft | Mostly the α-domain | 69A, 77R, 113R, | 65A, 73K, 109R, |
| | and a very small | 114A, 120C, 170R, | 110A, 116C, 166R, |
| | contribution from the | 206Q, 207P, 232R, | 202Q, 203P, 228R, |
| | β-domain | 285Y, 339R, 343R, | 281Y, 335R, 339R, |
| | | 357R, 392E, 394A, | 353R, 388E, 390S, |
| | | 452S, 629I, 642E | 448G, 625I, 638E |
| Substrate binding and | α-domain and | 113R (α-domain), | 109R (α-domain), |
| translocation | β-domain | 451N (β-domain) | 447N (β-domain) |
| Aromatic patch | α-domain | 161W, 162W, 220V | 157W, 158W, 216V |
| Active center | α-domain | 226N, 276H, 285Y | 222N, 272H, 281Y |
| Residues compensating | α-domain | 275Q, 339R, 343R, | 271Q, 335R, 339R, |
| for the catalytic | | 448E | 444E |
| residues | | | |
| Negative patch | α-domain | 265D, 275Q, 277S, | 261D, 271Q, 273S, |
| | | 448E | 444E |

TABLE 4
Major structural differences between HylA and
HylB, and crucial residues around the cleft

| | | Structural | |
|---|---|---|---|
| | | location | |
| HylA | HylB | (HylA) | Significance |
| 80T | 76E | | forms the negative cleft in HylB |
| 99N | 95D | near the cleft | forms the positive cleft in HylA |
| 106S | 102P | H3 | |
| 116S | 112E | H3 | forms the negative cleft in HylB |
| 132E | 128D (Side | near the cleft | forms the negative cleft in HylA |
| | chain) | | and HylB |
| 134H | 130E | near the cleft | forms the positive cleft in HylA |
| | | | and the negative cleft in HylB |
| 215H | 211N | near the cleft | forms the positive cleft in HylA |
| 221I | 217V | H7/H8 Loop | |
| 252Q | 248D | near the cleft | forms the negative cleft in HylB |
| 284S | 280G | H9/H10 Loop | |
| 309D | 305E | | forms the negative cleft in HylB |
| | (Cα-Cα | | |
| | distance) | | |
| 317S | 313D | | forms the negative cleft in HylB |
| 345D | 341N | | |
| 346E | 342G | H11/H12 Loop | |
| 361L | 357M | H12 | |
| 364N | 360D | Located at the | forms the negative cleft in HylB |
| | | center of the | |
| | | negative cleft: | |
| | | CRUCIAL | |
| 394A | 390S | H13/H14 Loop | |
| 395S | 391T | H13/H14 Loop | |
| 397R | 393V | | forms the positive cleft in HylA |
| 442N/S | 438D | B10/B11 Loop | Natural mutations in HylA |
| | | [H14/H15 | |
| | | Loop] | |
| 452S | 448G | B10/B11 Loop | |
| | | [H14/H15 | |
| | | Loop] | |
| 815S | 811D/H | | Natural mutations in HylB |
| | (793D/H) | | |

Comparison of HylA/B Structure with Other Bacterial Hyl Structures, Further Support Divergence of HylA/B

We were unable to get the crystals of HylA/B complexed with HA fragments. To get further insight into functional divergence of HylA/B, we compared structural features of HylA/B to other bacterial Hyls' structure from Streptococcus and Streptomyces species (FIG. 3a-d), and chondroitinases. Based on previous studies, Hyls are proposed to have evolved from the pre-existing chondroitinases. As Hyl enzymes evolved to recognize and process different substrates including HA, as expected, HylA/B share overall structural similarity to the Chondroitin AC lyase from Arthrobacter aurescens (ArthroAC; PDB: 1RW9) and to Hyl enzymes from other bacteria (Table 5). This observation suggests that HylA/B originated and functionally diverged from a common enzyme.

TABLE 5
Alignment of Hyalases crystal structures and
sequences from different bacterial species

| | | RMSD | Identity |
|---|---|---|---|
| | | (Å) from | (%) from |
| | | structural | sequence |
| Structure 1 | Structure 2 | alignment | alignment |
| HylA: | HylB: | 0.957 | 72.45 |
| Propionibacterium | Propionibacterium | | |
| acnes | acnes | | |
| | IN70: Hyl from | 8.54 | 21.34 |
| | Streptococcus pneumoniae | | |
| | 1F1S: Hyl from | 5.63 | 23.69 |
| | Streptococcus agalactiae | | |
| | 2X03: Hyl from | 4.87 | 30.03 |
| | Streptomyces coelicolor | | |
| | 1RW9: Chondroitin AC | 4.4 | 38.21 |
| | lyase | | |
| | Arthrobacter aurescens | | |
| | 2E24: Xanthan lyase from | 5.83 | 27.51 |
| | Bacillus sp. GL1 | | |

While the end-product profile of HylB (yielding only HA-2) is similar to the HA-degradation by Hyls from Streptococcus pneumoniae (SpHyl), Streptococcus agalactiae (SaHyl) and Streptomyces coelicolor (ScHyl), end-products from HylA contain both larger fragments and disaccharides. Mechanistically, HA degradation by chondroitinases and Hyl enzymes follow two types of mechanisms. Based on the product profile, HylB exploits the processive/progressive exolytic cleavage mechanism. We suggest that HylA follows a two-step process that initially involves the non-processive random bite endolytic cleavage and later adopt exolytic functionality. The observation that HylA cleft is more open than the HylB cleft (Tables 6 and 7) is in correlation with the above inference of mechanistic differences in their HA-degradation. Thus, combined, the data suggest that HylA and HylB functionally diverged by switching from endolytic to exolytic processing for efficient degradation of HWA-HA concomitant with different biological effects on host.

TABLE 6
Comparison of the cleft conformations among bacterial Hyls.

| | Apo | Complex | Apo | Complex |
|---|---|---|---|---|
| | structures | structures | structures | structures |
| | with Open | with Open | with Closed | with Closed |
| | conformation | conformation | conformation | conformation |
| Bacteria/Enzyme | of the cleft | of the cleft | of the cleft | of the cleft |
| Propionibacterium | HylA; HylB | | | |
| acnes Hyl (PaHyl) | | | | |
| Streptomyces | 2X03 | | | 2WCO; 2WDA |
| coelicolor Hyl (ScHyl) | | | | |
| Streptococcus | 2BRW | | 2BRV; 1EGU | 1C82; 1LXK; |
| pneumoniae Hyl (SpnHyl) | | | | 1LOH; 1F9G |
| Streptococcus | 1F1S | 118Q; 1LXM | | |
| agalactiae Hyl (SaHy1) | | | | |

TABLE 7
The closest distances around the active site cleft depicting
the extent of openness of the active site cleft

| | | | | | | Closest |
|---|---|---|---|---|---|---|
| | | | | | | distance |
| | | | | | | between |
| | | | | | Ca—Ca | side |
| | | | | | distance | chains |
| Structure | Feature 1 | Residue 1 | Feature 2 | Residue 2 | (Å) | (Å) |
| HylA | H4/H5 | 160N | B10/B11 | 451N | 18.2 | 12.0 |
| (Propionibacterium | Loop | | Loop | | | |
| acnes) | | | [H14/H15 | | | |
| | | | Loop] | | | |
| HylB | | 156N | | 447N | 16.6 | 10.6 |
| (Propionibacterium | | | | | | |
| acnes) | | | | | | |
| 1LOH | | 290N | | 580N | 9.2 | 3.6 |
| (Streptococcus | | | | | | |
| pneumoniae) | | | | | | |
| 1LXM | | 370N | | 660N | 15.3 | 9.5 |
| (Streptococcus | | | | | | |
| agalactiae) | | | | | | |
| 2WCO | | 139N | | 432N | 8.6 | 3.4 |
| (Streptomyces | | | | | | |
| coelicolor) | | | | | | |

### Proposed HylA/B Degradation Mechanisms and Relation to Position of Amino Acid Residue Changes

To understand the structural basis for the differences in HA-degradation in relation to the amino acid changes in HylA/B, based on previous studies, we have categorized residues into 3 groups: (1) residues involving in the basic catalysis [residues forming the active center], (2) residues involving in substrate binding/positioning, and product release [residues forming the positive cleft, aromatic and negative patches], and (3) residues involving in the regulation of substrate entry and translocation/sliding [the residues involving in the domain movements and structural flexibility]. We assessed several of these residues for their activity through point mutations (FIG. 13).

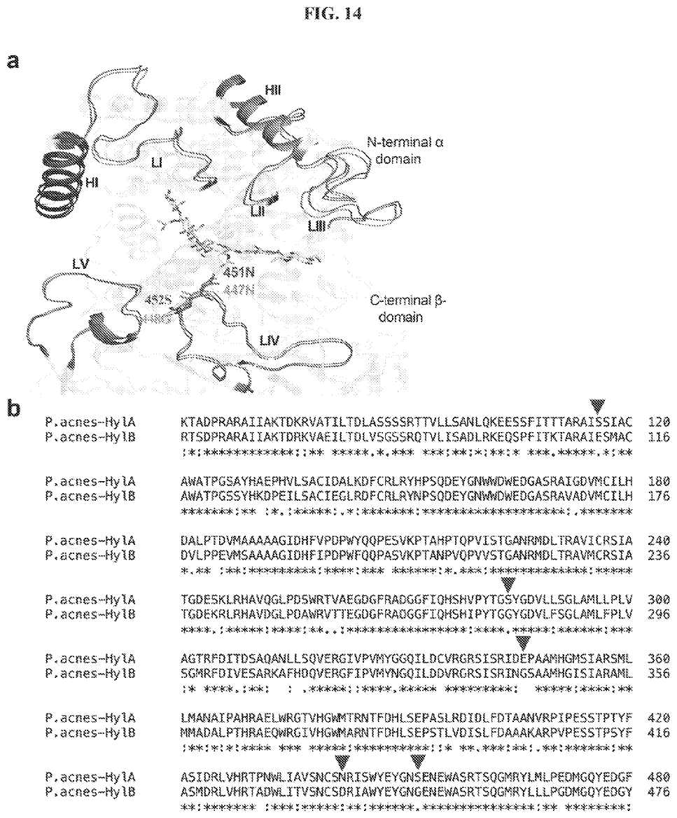

Since the residues from group 1 are highly conserved in HylA/B and involved in basic catalysis, we have chosen their surrounding residues, including 116S/112E, and 284S/280G (HylA/B numbering), among few others. From group 2, significant differences are observed in the residues forming the positive patch (charged cleft) including 346E/342G and 397R/393V, among few others. In addition to the above differences, inter-domain motions in SpHyl, SaHyl and ScHyl enzymes have been implicated in substrate processing. Based on this notion, we have identified the loops and helices associated with these functions (FIG. 3c and FIG. 14a) and the major differences in HylA/B, which include 346E/342G, 394A/390S, 395S/391T, 442N/438D, and 452S/448G (FIG. 14b). These differences critically alter the HA degradation mechanism, as the residues involving in the domain movements and structural flexibility can regulate the substrate entry and translocation/sliding between the subsequent catalytic cycles of the processive degradation of the polymeric/oligomeric HA substrate. It is possible that interdomain movements in HylA allow the enzyme to initially engage in endolytic activity and gradually switch to exolytic activity depending on the size of the substate available.

### Single Amino Acid, Serine 452 Located Outside the Substrate Binding Domain, Drives the Divergence of Enzymatic Mechanisms

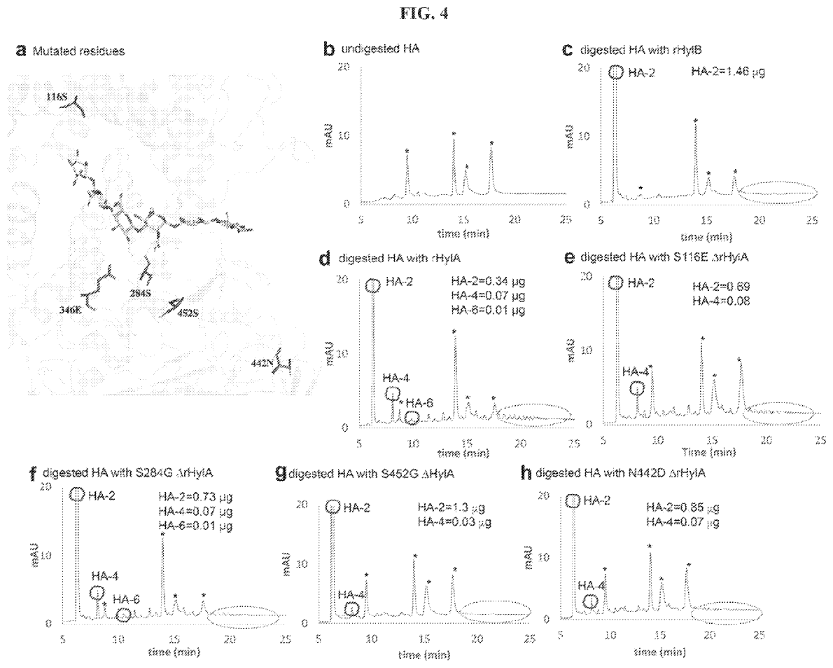

We performed point mutations in several of the residues in HylA, based on the potential effects of specific HylA/B residues on HA degradation (FIG. 4a-h and FIG. 14b).

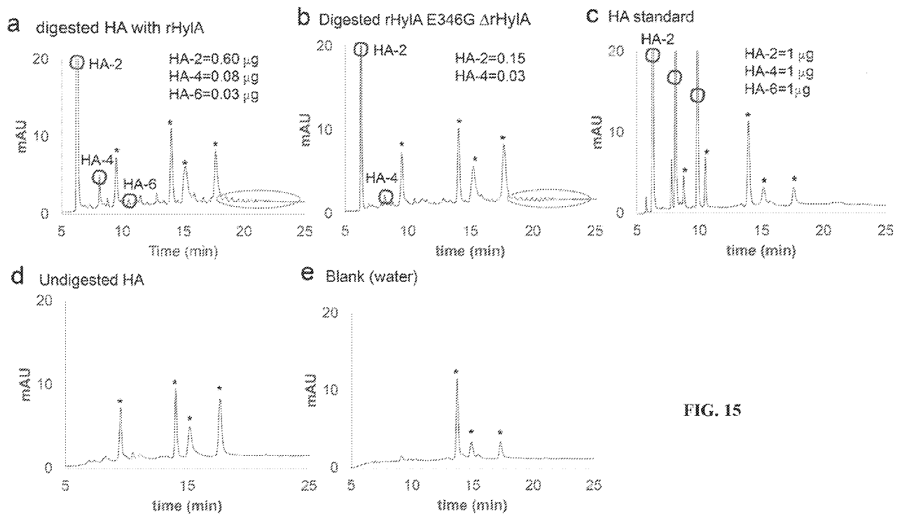

Remarkably, HylA mutant S452G significantly reversed enzymatic phenotype to HylB (FIG. 4g). The mutant protein digested the HMW-HA substrate to HA-2 (phenocopying the WT HylB) with trace residual amounts of HA-4. Other point mutations, in particularly 442N, accelerated the digestion of HA to HA-2 and decreased the amounts of undigested HA (FIG. 4h and FIG. 15).

Structurally, 452S and 442N residues are located in the loop LIV from the β-domain (FIG. 14a). Change at this location reversed the phenotype possibly by affecting substrate positioning and enzyme's activity. Joshi et al have compared the role of interdomain motions in other Hyl homologs. Briefly, their analyses show that the corresponding residue of 451N (HylA), (preceding residue of 452S (HylA)) in SpHylA (580N) was shown to involve in substrate binding/translocation and the loop harboring this residue (βLIII in SpHyl and LIV in Hyl) control the substrate entry. Mutation of 580N to Glycine in SpHyl was also shown to alter its enzymatic activity by further opening of the cleft (domain motion (i)). Hence, replacing Ser to Gly in HylA at this location is likely to impart higher flexibility in loop LIV and increase the domain motion. Of note, the exolytic ScHyl (PDB: 2WCO) and ArthroAC (PDB: 1RW9) have Glycine at this location suggesting that HylB might have acquired efficient way to process HA through interdomain motions.

These observations suggest that HylA may have diverged into more efficient HylB by regulating substrate's entry, binding, and translocation/sliding through interdomain motions.

### Proinflammatory Properties of Hyl Degradation Products

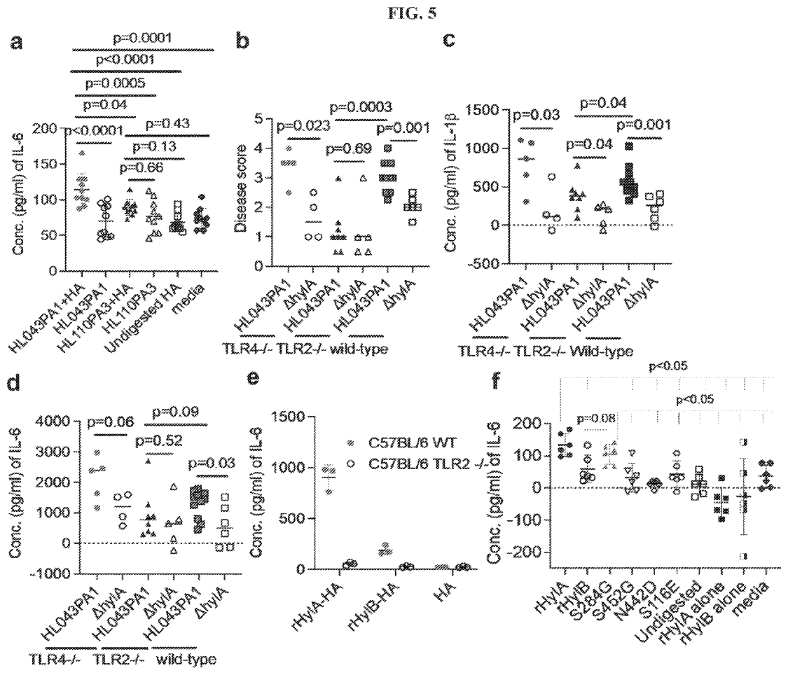

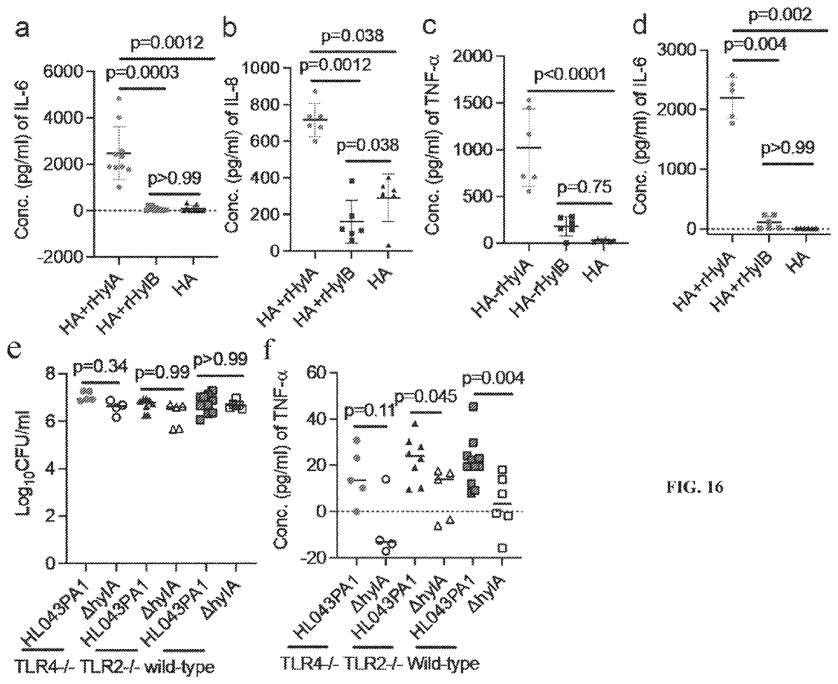

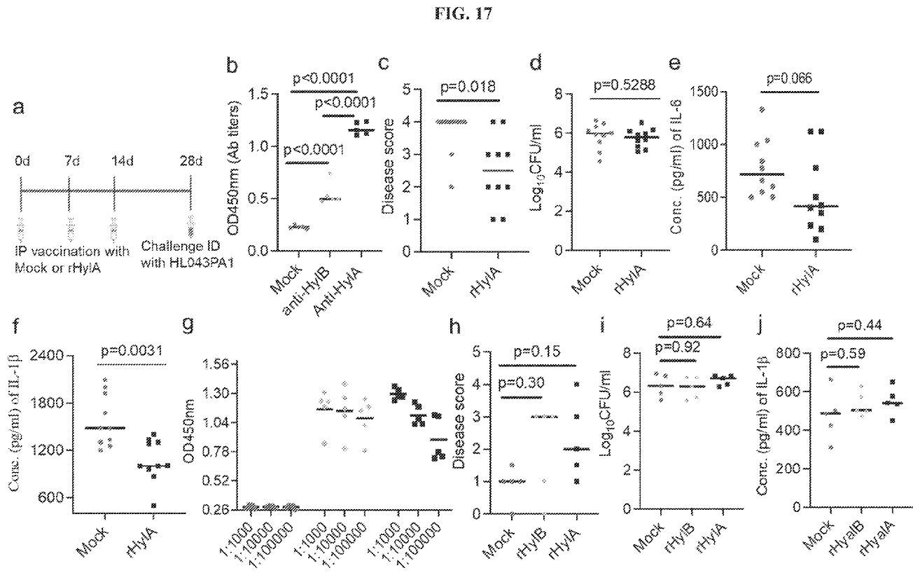

Having defined the structures and enzymatic functions of C. acnes Hyls, we asked if the HA products of degradation from HylA and HylB induce inflammatory pathology noted in the in vivo experiments. For these assays, we digested HA with rHyls (FIG. 16a-d) or supernatant derived from WT/ΔhylA (FIG. 5a) for 24 h and measured cell-specific cytokine secretion responses. We showed that HylA induced higher levels of acne-related cytokines in keratinocyte (FIG. 5a and FIG. 16a,b) and macrophage cell lines (Extended data FIG. 16c,d) than controls, irrespective of whether supernatant or higher concentration of rHylB is used. In comparison, HylB degradation induced reduced (IL-8) or no change in other cytokine levels compared to controls, consistent with the previously defined anti-inflammatory properties of processive HA-2 producing Hyl enzymes. As reported, HA-2 lacks pro-inflammatory property, and degradation of HA to HA-2 abrogates proinflammatory properties of the larger HA fragments. Furthermore, HA-2 competes with the larger-sized HA to further block TLR2 activation.

The TLR2 dependence of acne vulgaris is a well-recognized feature of the skin disease, and therefore we interrogated TLR2 dependence of inflammation induced by HylA/B. Consistent with the TLR2 dependence of HA (FIG. 5b-e), difference in pathology induced by HL043PA1 and ΔhylA is abrogated in TLR2^{-/-} but not in TLR4 mice.

Finally, we asked how the single amino acid substitutions that modified or reversed Hyl degradation impacted inflammation. As shown in FIG. 5f and consistent with the product of the Hyl HA degradation, single amino acid substitution (S452G) that reverted or accelerated processing of HA also led to reversion of the inflammatory phenotype. We also noted reversal in inflammation by several other HylA single amino acid mutants, which correlated with the higher HA-2 and lower amounts of undigested larger oligos they produce compared to WT rHylA.

Overall, our findings are consistent with Hyl generation of distinct degradation product sizes that leads to different inflammatory outcomes. The potential importance of Hyl in humans is further advanced by linking Hyl mechanisms and acne through their TLR2 dependence.

### Targeting Hyl to Treat Acne Disease

Above, we have shown that HylA plays a major role in the immunopathology of acne in our murine model. HylA is highly conserved with consistent enzymatic activity demonstrated across phylotypes of C. acnes. Hence, it is a good target for therapeutic intervention. Alternatively, the significant homology between HylA and HylB poses a potential challenge of therapeutic selectivity.

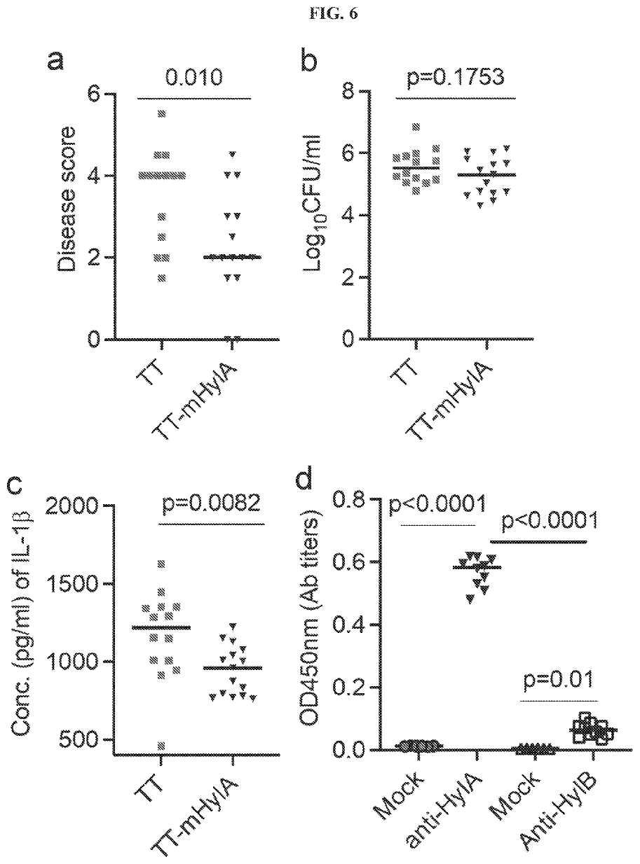

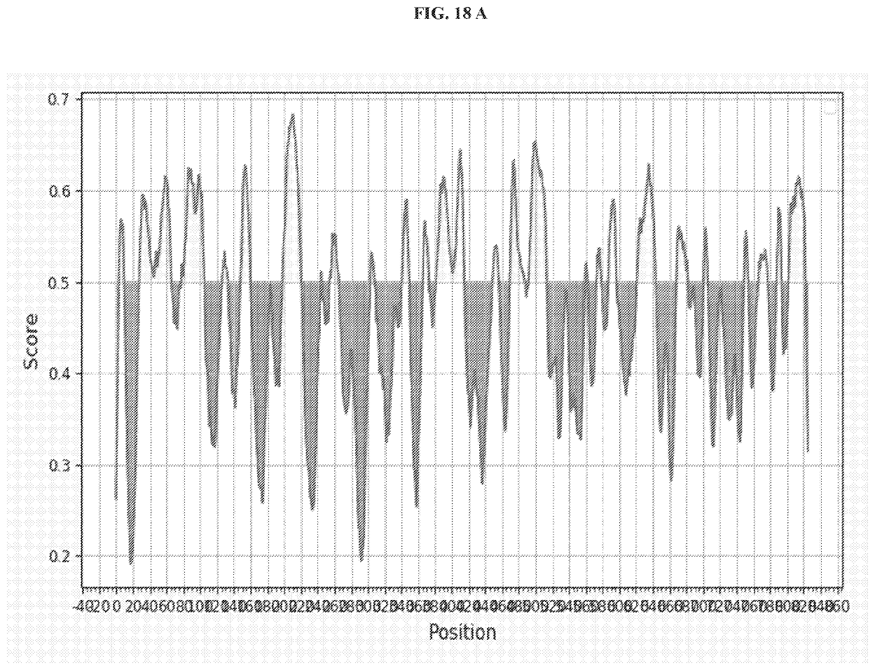

To begin, we tested if immunization against HylA conferred protection against acne. We injected mice three times at one-week intervals, then challenged the mice with HL043PA1 FIG. 17a). HylA vaccination in alum induced robust antibody response against HylA (FIG. 17b) and significantly reduced immunopathology associated with murine acne disease (FIG. 6a,b and FIG. 17c-d). However, HylA (FIG. 17b) or HylB (FIG. 17e) immunization induced cross-reactive antibodies to each other, and modest deterioration of disease score when the mice were challenged with health-associated HL110PA3 strain (FIG. 17f-h).

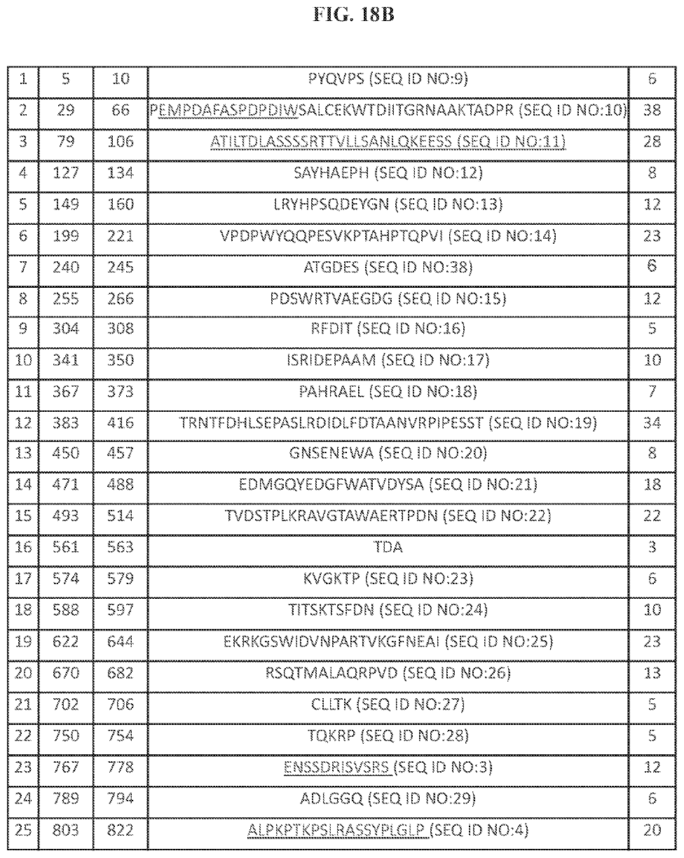

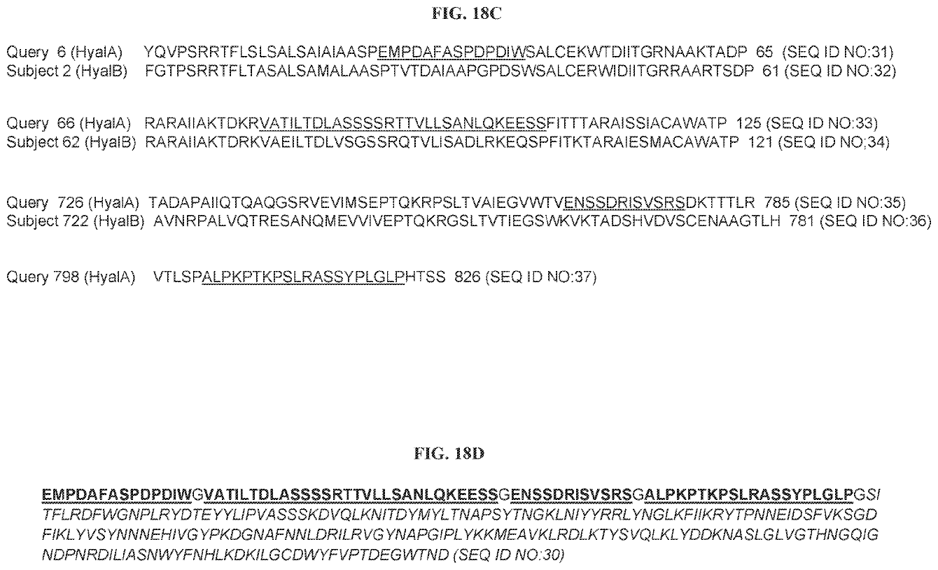

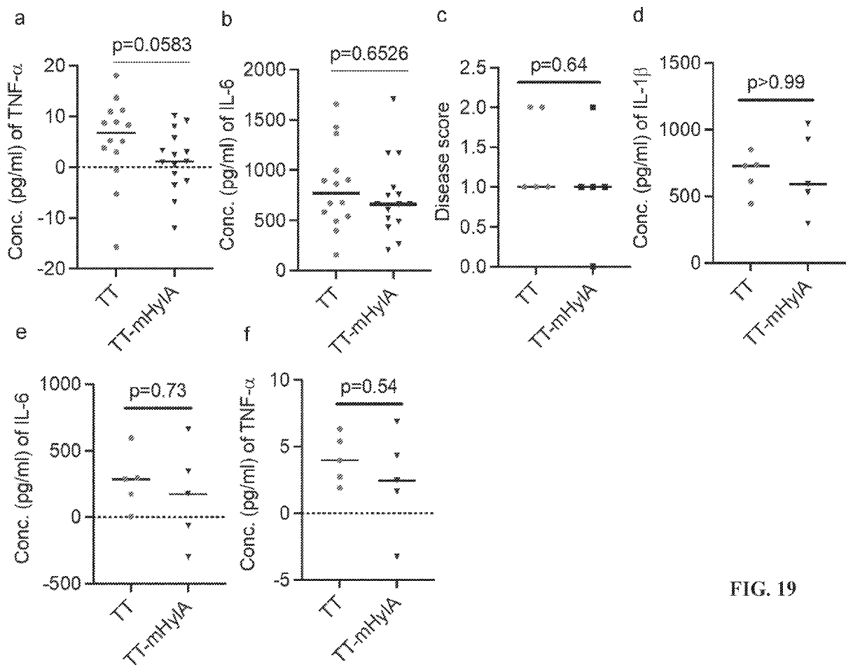

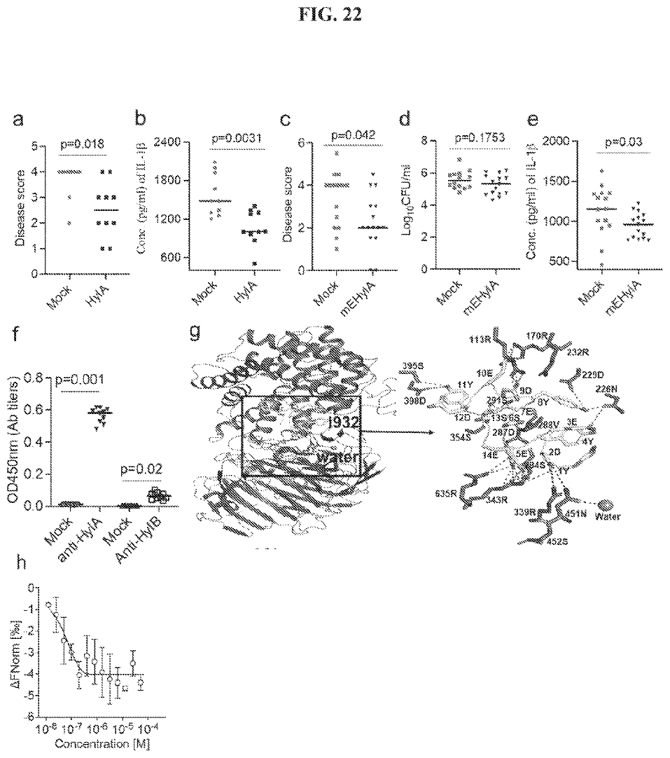

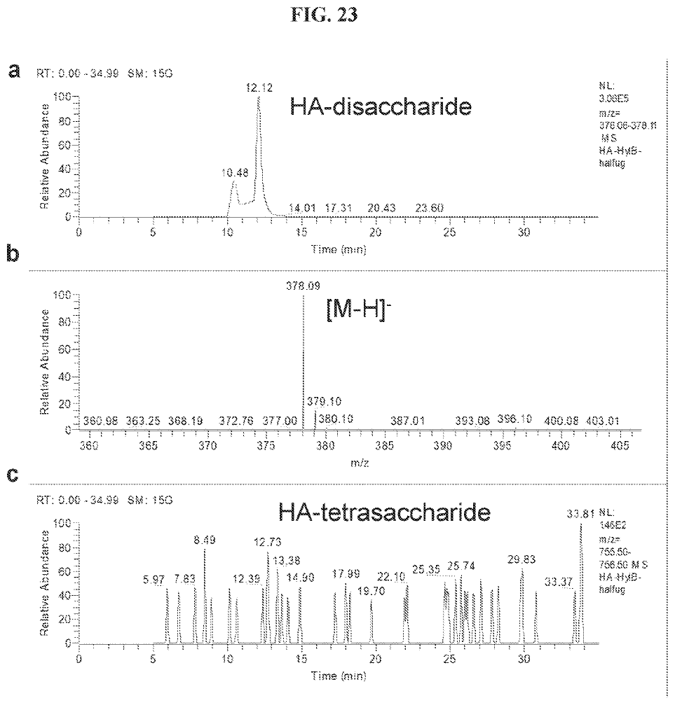

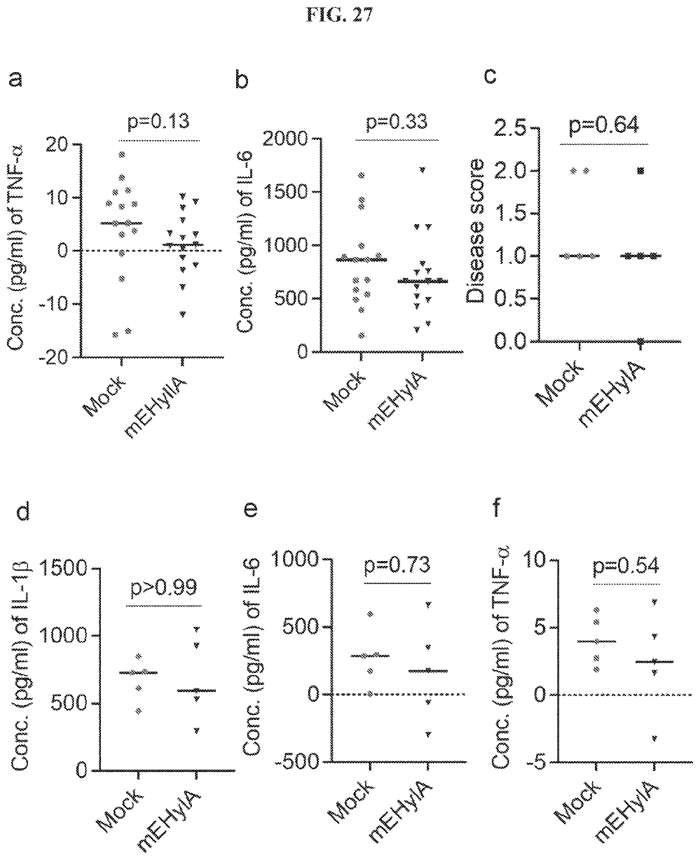

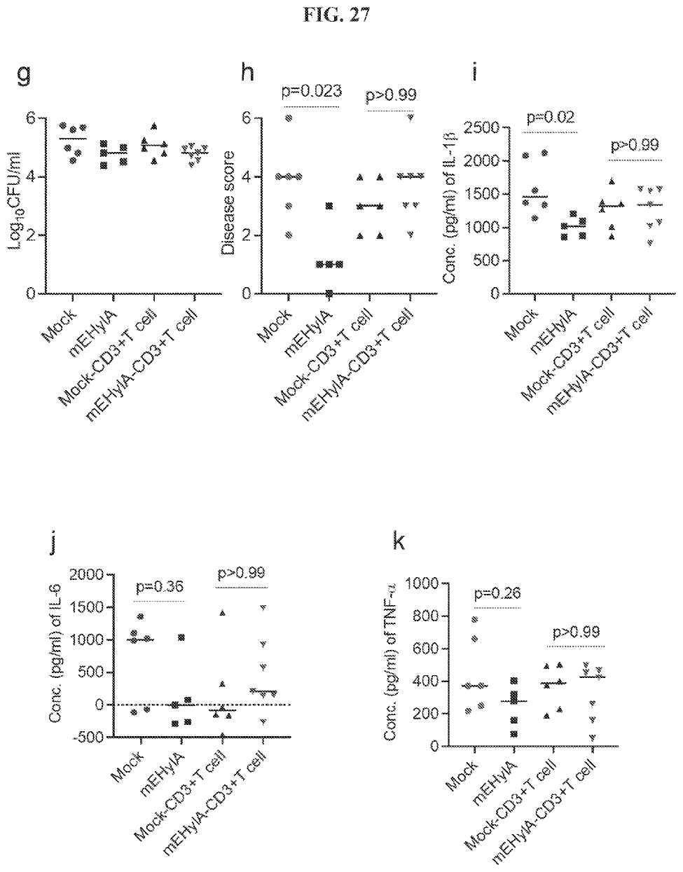

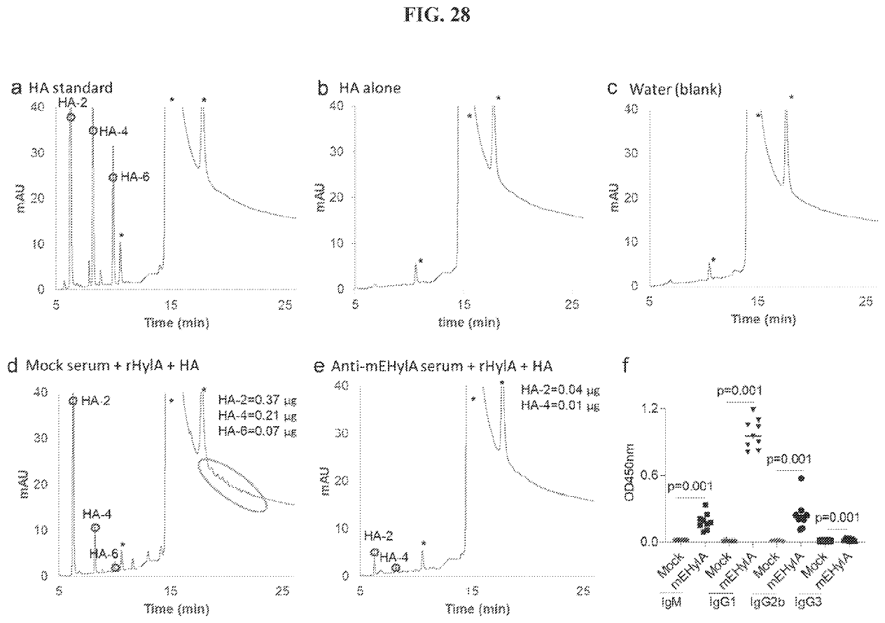

To circumvent the potential for inducing inflammation related to cross-reactive antibodies, we designed a HylA-specific peptide vaccine using the combination of antigenicity program (IEDB analysis resources, tools.iedb.org/bcell/) (FIG. 18a-d) and the crystal structures of HylA and HylB. The peptide combines several HylA-specific epitopes and has no significant homology to human proteins. We physically linked the predicted peptides to tetanus toxoid (TT), expressed the fusion protein in E. coli, and then verified the construct by mass-spectrometry. The vaccine selectively inhibited acne caused by HL043PA1 (FIG. 22c-e) with minimal evidence of inflammation from cross-reactivity to HylB from HL110PA3 (FIG. 22f and FIG. 27a-f). In an adoptive T cell transfer experiment, CD3⁺T cells from vaccinated mice were shown to be dispensable for protection against acne disease (FIG. 27g-k). Next, we tested the effect of post-mEHylA vaccination serum on rHylA enzymatic activity. Post vaccination serum significantly reduced HA degradation by HylA enzyme (FIG. 28a-e). Notably, mEhylA generated predominant IgG1 anti-HylA antibodies (FIG. 28f).

Accordingly, various embodiments of the present invention are based, at least in part, on these findings.

### Polypeptides

Various embodiments of the present invention provide for a polypeptide, comprising: a fragment of HylA. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 5-9, 10-14, 15-20, 21-25, 26-30, 31-35, or 36-40 amino acid residues in length. In various embodiments, the fragment of HylA has the amino acid sequence of any one of SEQ ID NO:9-29 and 38.

Various embodiments of the present invention provide for a polypeptide, comprising: a fragment of HylA linked or fused to an adjuvant. In various embodiments, the adjuvant is a polypeptide adjuvant. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 5-9, 10-14, 15-20, 21-25, 26-30, 31-35, or 36-40 amino acid residues in length. In various embodiments, the fragment of HylA has the amino acid sequence of any one of SEQ ID NO:9-29 and 38.

In various embodiments, the fragment of HylA comprises one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 2 or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the adjuvant is a tetanus protein, pertussis toxoid, diphtheria toxoid, or a fragment thereof.

In various embodiments, the polypeptide further comprises a linker between the one or more peptides and the adjuvant. The linker can be a peptide linker.

In various embodiments, the polypeptide comprises at least two peptides and further comprises a linker between each of the at least two peptides.

In various embodiments, the linker is G, polyserine, polyglycine, GGGGS (SEQ ID NO:5) or GGGGGS (SEQ ID NO:6), leucine zipper, or aliphatic.

Various embodiments of the present invention provide for a polypeptide, comprising: one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 2 or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the one or more peptides is linked or fused to an adjuvant. In various embodiments, the adjuvant is a polypeptide adjuvant.

In various embodiments, the adjuvant is a tetanus protein, pertussis toxoid, diphtheria toxoid, or a fragment thereof.

In various embodiments, the polypeptide further comprises a linker between the one or more peptides and the adjuvant. The linker can be a peptide linker. In various embodiments, the polypeptide further comprises a linker between the one or more peptides and N- or C-terminus of the adjuvant.

In various embodiments, the polypeptide comprises at least two peptides and further comprises a linker between each of the at least two peptides.

In various embodiments, the linker is G, polyserine, polyglycine, glycine-serine, GGGGS (SEQ ID NO:5) or GGGGGS (SEQ ID NO:6), leucine zipper, r aliphatic, or helical peptides.

Various embodiments provide for a composition comprising a polypeptide and an adjuvant.

In various embodiments, the polypeptide is a fragment of HylA. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 5-9, 10-14, 15-20, 21-25, 26-30, 31-35, or 36-40 amino acid residues in length. In various embodiments, the fragment of HylA has the amino acid sequence of any one of SEQ ID NO:9-29 and 38.

In various embodiments, the polypeptide comprises: one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 2 or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the adjuvant is alum, hydroxyphosphate sulfate, CpG1018, monophosphoryl lipid A, oil-in-water emulsion, CpG, or QS-21 saponin.

In various embodiments, the polypeptide further comprises 1-10 amino acid residues on the N-terminus, the C-terminus, or both. In various embodiments, the peptide further comprises 1-5 amino acid residues on the N-terminus, the C-terminus, or both. In various embodiments, the peptide further comprises 1-3 amino acid residues on the N-terminus, the C-terminus, or both.

In various embodiments, the polypeptide comprises L-amino or D-amino acid and/or equivalent non-natural amino acids

In various embodiments, the polypeptide comprises alpha- or beta-amino acids

In various embodiments, the polypeptide comprises non-hydrolyzable bonds.

Various embodiments provide for mRNAs encoding the polypeptides of the present invention. Embodiments, also provide for mRNA compositions for eliciting an immune response. mRNA compositions (e.g., immune compositions, vaccines) work by introducing a piece of mRNA that encode the polypeptide. By using this mRNA, cells can produce the polypeptide. As part of a normal immune response, the immune system recognizes that the polypeptide is foreign and produces antibodies.

Briefly, in vitro transcribed mRNA can be produced from a linear DNA template using a T7, a T3 or an Sp6 phage RNA polymerase. The resulting product should optimally contain an open reading frame that encodes the protein of interest, flanking UTRs, a 5′ cap and a poly(A) tail. The mRNA is thus engineered to resemble fully processed mature mRNA molecules as they occur naturally in the cytoplasm of eukaryotic cells.

In vitro and in vivo transfection reagents have been developed that facilitate cellular uptake of mRNA and protect it from degradation. Once the mRNA transits to the cytosol, the cellular translation machinery produces protein that undergoes post-translational modifications, resulting in a properly folded, fully functional protein or polypeptide. This feature of mRNA pharmacology is particularly advantageous for vaccines and protein replacement therapies that require cytosolic or transmembrane proteins to be delivered to the correct cellular compartments for proper presentation or function.

Various embodiments of the invention provide for a pharmaceutical composition comprising any one of the immunogenic peptides of the present invention as described herein, or mRNAs encoding the immunogenic peptides of the present invention as described herein. In various embodiments, the compositions are in the form of nanoparticles.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective amount can be as indicated to the skilled artisan by the in vitro responses or responses in animal models. Such dosages typically can be reduced by up to about one order of magnitude in concentration or amount without losing the relevant biological activity. Thus, the actual dosage will depend upon the judgment of the physician, the condition of the patient, and the effectiveness of the therapeutic method based, for example, on the in vitro responsiveness of the relevant primary cultured cells or histocultured tissue sample, the responses observed in the appropriate animal models, as previously described.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal or parenteral.

“Transdermal” administration may be accomplished using a topical cream or ointment or by means of a transdermal patch.

“Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may also be in the form of microspheres or nanospheres or lipid vesicles or polymer vesicles allowing controlled release

Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release.

Via the topical route, the pharmaceutical compositions based on compounds according to the invention may be formulated for treating the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication.

Additionally, the peptides of the present invention can be linked to nano-adjuvants. For example, the peptides of the present invention can be linked to polymersomes (polymer vesicles self-assembled from a diverse array of synthetic amphiphilic block copolymers containing hydrophilic and hydrophobic blocks), as described by Levine et al. Methods 46 (2008) 25-32. The peptides of the present invention can also be conjugated to nanoparticles, such as albumin, liposomes, polymers, gold nanoparticles, as well as iron oxide nanoparticles such as those described in Mu et al., Nanoscale. 2015 Nov. 21; 7(43): 18010-18014. These publications are herein incorporated by reference as though fully set forth herein.

### Kits

The present invention is also directed to a kit to treat acne or reduce the likelihood of acne, or inhibit C. acnes hyaluronidase. The kit is useful for practicing the inventive methods of treating acne or reducing the likelihood of acne, or inhibiting C. acnes hyaluronidase. The kit is an assemblage of materials or components, including at least one of the inventive polypeptides or compositions. Thus, in some embodiments the kit contains a composition including any one or more of the immunogenic peptides of the present invention, as described above.

The exact nature of the components configured in the inventive kit depends on its intended purpose. In various embodiment, the kit is configured particularly for the purpose of treating mammalian subjects. In various embodiment, the kit is configured particularly for the purpose of treating human subjects.

Instructions for use may be included in the kit. “Instructions for use” typically include a tangible expression describing the technique to be employed in using the components of the kit to effect a desired outcome, such as to treat acne or reduce the likelihood of acne, or inhibit C. acnes hyaluronidase. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing any one or more of the immunogenic peptides of the present invention as described herein. The packaging material generally has an external label which indicates the contents and/or purpose of the kit and/or its components.

### Methods

Various embodiments provide for a method of treating acne, comprising: administering a polypeptide of the present invention as described herein or a composition of the invention as described herein to a subject in need thereof.

Various embodiments provide for a method of reducing the likelihood of acne, comprising: administering a polypeptide of the present invention as described herein or a composition of the invention as described herein to a subject in need thereof.

Various embodiments provide for a method of eliciting an immune response in a subject in need thereof, comprising: administering a polypeptide of the present invention as described herein or a composition of the invention as described herein to a subject in need thereof. In various embodiments, the immune response is a protective immune response.

Various embodiments provide for a method of treating acne, comprising: administering a composition comprising an mRNA molecule encoding a polypeptide of the present invention as described herein to a subject in need thereof. Various embodiments provide for a method of reducing the likelihood of acne, comprising: administering a composition comprising an mRNA molecule encoding a polypeptide of the present invention as described herein to a subject in need thereof.

Various embodiments provide for a method of eliciting an immune response in a subject in need thereof, comprising: a composition comprising an mRNA molecule encoding a polypeptide of the present invention to a subject in need thereof. In various embodiments, the immune response is a protective immune response.

As such, in various embodiments, the polypeptide comprises: a fragment of HylA. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 10-14, 15-20, 21-25, 26-30, or 31-35 amino acid residues. In various embodiments, the fragment of HylA has the amino acid sequence of any one of SEQ ID NO:9-29 and 38.

Also as such, in various embodiments, the polypeptide comprises: a fragment of HylA linked or fused to an adjuvant. In various embodiments, the adjuvant is a polypeptide adjuvant. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 10-14, 15-20, 21-25, 26-30, or 31-35 amino acid residues. In various embodiments, the fragment of HylA has the amino acid sequence of any one of SEQ ID NO:9-29 and 38.

In various embodiments, the fragment of HylA comprises one or more peptides selected from
(a) EMPDAFASPDPDIW (SEQ ID NO:1),
(b) a EMPDAFASPDPDIW (SEQ ID NO:1) variant, the EMPDAFASPDPDIW

(SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 2 or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the adjuvant is a tetanus protein, pertussis toxoid, diphtheria toxoid, or a fragment thereof.

In various embodiments, the polypeptide further comprises a linker between the one or more peptides and the adjuvant. The linker can be a peptide linker In various embodiments, the polypeptide further comprises a linker between the one or more peptides and N- or C-terminus of the adjuvant.

In various embodiments, the polypeptide comprises at least two peptides and further comprises a linker between each of the at least two peptides.

In various embodiments, the linker is G, polyserine, polyglycine, glycine-serine, GGGGS (SEQ ID NO:5), GGGGGS (SEQ ID NO:6), leucine zipper, r aliphatic, or helical peptides.

As such in other embodiments, the polypeptide used in these methods comprises: one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 2 or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the one or more peptides is linked or fused to an adjuvant. In various embodiments, the adjuvant is a polypeptide adjuvant.

In various embodiments, the adjuvant is a tetanus protein, pertussis toxoid, diphtheria toxoid, or a fragment thereof.

In various embodiments, the further comprises a linker between the one or more peptides and the adjuvant. The linker can be a peptide linker In various embodiments, the polypeptide further comprises a linker between the one or more peptides and N- or C-terminus of the adjuvant.

In various embodiments, the polypeptide comprises at least two peptides and further comprises a linker between each of the at least two peptides.

In various embodiments, the linker is G, polyserine, polyglycine, glycine-serine, GGGGS (SEQ ID NO:5), GGGGGS (SEQ ID NO:6), leucine zipper, r aliphatic, or helical peptides.

As such in yet other embodiments, the composition used in these methods comprises a polypeptide and an adjuvant,

In various embodiments, the polypeptide is a fragment of HylA. In various embodiments, the fragment of HylA is about 12-29 amino acid residues. In various embodiments, the fragment of HylA is about 10-31 amino acid residues, 9-32 amino acid residues, or 8-33 amino acid residues. In various embodiments, the fragment of HylA is about 5-9, 10-14, 15-20, 21-25, 26-30, 31-35, or 36-40 amino acid residues in length. In various embodiments, the fragment of HylA has the amino acid sequence of any one of SEQ ID NO:9-29 and 38.

In various embodiments, the polypeptide comprises: one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

In various embodiments, the EMPDAFASPDPDIW (SEQ ID NO:1) variant has up to 6, 5, 4, 3, 2, or 1 amino acid additions, substitution, or deletions. In various embodiments, the EMPDAFASPDPDIW (SEQ ID NO:1) variant has up to 3, 2, or 1 amino acid additions, substitution, or deletions.

In various embodiments, the VATILTDLASSSSRTTVLLSANLQKEESS (SEQ ID NO:2) variant, has up to 16, 15, 14, 13, 12, 11, 10, 9, 8, 7, 6, 5, 4, 3, 2, or 1 amino acid additions, substitution, or deletions. In various embodiments, the VATILTDLASSSSRTTVLLSANLQKEESS (SEQ ID NO:2) variant, has up to 8, 7, 6, 5, 4, 3, 2, or 1 amino acid additions, substitution, or deletions.

In various embodiments, the ENSSDRISVSRS (SEQ ID NO:3) variant has up to 5, 4, 3, 2, or 1 amino acid additions, substitution, or deletions. In various embodiments, the ENSSDRISVSRS (SEQ ID NO:3) variant has up to 3, 2, or 1 amino acid additions, substitution, or deletions.

In various embodiments, the ALPKPTKPSLRASSYPLGLP (SEQ ID NO:4) variant has up to 9, 8, 7, 6, 5, 4, 3, 2, or 1 amino acid additions, substitution, or deletions. In various embodiments, the ALPKPTKPSLRASSYPLGLP (SEQ ID NO:4) variant has up to 4, 3, 2, or 1 amino acid additions, substitution, or deletions.

In various embodiments, the variant has at least 60%, 70%, 80%, 90%, 95%, 96%, 97%, 98%, or 99% sequence identity to its reference polypeptide. In various embodiments, the variant has at least 95%, 96%, 97%, 98%, or 99% sequence identity to its reference polypeptide. In various embodiments, the variant has a linear epitope score of at least 0.5, 0.6, 0.7, 0.8 or 0.9 to its reference polypeptide. In various embodiments, the variant has a linear epitope score of at least 0.9 to its reference polypeptide.

In various embodiments, the adjuvant is alum, hydroxyphosphate sulfate, CpG1018, monophosphoryl lipid A, oil-in-water emulsion, CpG, or QS-21 saponin.

In various embodiments, the polypeptide further comprises 1-10 amino acid residues on the N-terminus, the C-terminus, or both. In various embodiments, the peptide further comprises 1-5 amino acid residues on the N-terminus, the C-terminus, or both. In various embodiments, the peptide further comprises 1-3 amino acid residues on the N-terminus, the C-terminus, or both.

In various embodiments, the polypeptide comprises L-amino or D-amino acid and/or equivalent non-natural amino acids

In various embodiments, the polypeptide comprises alpha- or beta-amino acids

In various embodiments, the polypeptide comprises non-hydrolyzable bonds.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

C. acnes Bacterial Culture

Two acne-associated strains (HL043PA1 and HL043PA2) and two health-associated strains (HL110PA3 and HL110PA4) were used in this study. Clinical C. acnes strains from frozen stock were cultured on blood agar plates anaerobically using BD BBL™ GasPak™ system for 96 h at 37° C. A single colony of C. acnes was anaerobically grown in 10 ml of Brain Heart Infusion (BHI) broth (Catalog no. #53286, Sigma-Aldrich, USA) for 3-4 days (OD=0.15-0.3), followed by once washing of the bacterial pellet with BHI media at 2300×g for 5 min. The pellet was resuspended in BHI media to a desired OD_{600 nm} (0.5) for in vitro and in vivo studies. Bacterial culture supernatant was collected and used for rooster comb HA (Catalog no. #H5388, Sigma-Aldrich, USA) degradation activity, sodium dodecyl sulfate-polyacrylamide gel electrophoresis (SDS-PAGE) analysis, and human keratinocyte HaCaT cell (ATCC) stimulation.

Construction of ΔhylA and ΔhylB C. acnes Strains

The homologous recombination cloning strategy performed was previously described (Sorensen et al., 2010) with slight modifications. Briefly, 1 kb (500 bp) up and down stream of the hyaluronidase gene was amplified by PCR, gel purified, ligated together and cloned into pGEM-T-easy (catalog #A137A, Promega, USA). The erythromycin resistance cassette from pDCerm was PCR amplified and ligated between the up and down stream regions before being transformed into E. coli (DH5α) (catalog #18265017, ThermoFisher Scientific). Plasmid DNA from ampicillin (100 μg/mL) resistant clones was purified and verified by PCR. Correct plasmids were transformed into dam-negative E. coli (Catalog #C2925I, New England Biolabs) and purified. Competent C. acnes cells were prepared as described previously (Cheong et al., 2008). Briefly, C. acnes was grown in BHI medium to an OD_{600 nm} of 0.5-0.6 anaerobically at 37° C. Cells were pelleted and washed in EP buffer (272 mM sucrose, 7 mM sodium phosphate, and 1 mM magnesium chloride) twice. Plasmid DNA was mixed with freshly made electrocompetent C. acnes cells and electroporated. One mL of BHI was immediately added following electroporation. The cells were pelleted and resuspended in 100 μl of BHI medium, plated onto BHI agar plates and incubated at 37° C. anaerobically overnight. The next day the bacteria were removed with a cotton swab, placed in fresh BHI medium, plated onto BHI plates containing erythromycin (10 μg/ml) (Catalog no. #E5389, Sigma-Aldrich) and incubated at 37° C. until colonies appeared (5-7 days). Mutants were verified by PCR and the lack of activity confirmed on agar plates containing hyaluronan.

### Hyaluronidase Plate Assay

Hyaluronate lyase activity of HylA or HylB in C. acnes culture supernatants was measured using HA from rooster comb as a substrate. 20 μL or 40 μL of supernatant from a single colony of C. acnes, grown anaerobically for 4 days and harvested at 2600×g for 10 min, was spotted on BHI agar plates containing 1% bovine serum albumin (BSA) fraction V (Catalog no. #10735078001, Sigma-Aldrich, USA) and HA (400 μg/mL). The plates were incubated at 37° C. overnight, and HA degradation was detected by flushing the plate with 2N acetic acid for 3-5 min.

### Cell Cultures

Bone marrow derived macrophages (BMDMs) were isolated from the femurs and tibiae of 12-week-old C57BL/6 mice (Jackson laboratories) and suspended in complete RPMI 1640 media (Gibco, ThermoFisher Scientific, USA) with 10% heat-inactivated fetal bovine serum (FBS), 10 ng/ml of M-CSF (Catalog no. #PeproTech, Inc., USA) and 1% of Penicillin-Streptomycin antibiotics (Catalog #P4333, Sigma-Aldrich, St. Louis, MO, USA). Cells were cultured in 92 mm non-adherent dishes (ThermoFisher Scientific, USA) at 37° C. under 5% CO₂, followed by the replacement of media with the fresh media containing equivalent concentrations of M-CSF every other two days. Then, seven days post-culture, cells were harvested and stimulated with HA (40 μg) that was digested with either bacterial supernatant or rHylA or rHylB enzymes.

HaCaT cells (ATCC) were cultured in complete DMEM media (Catalog no. 10-013-CV, Corning incorporated, USA) plus 10% heat-inactivated FBS in 5% CO₂ at 37° C. Before cell stimulation with digested HA (40 μg), HaCaT cells were seeded in 96-wells Falcon® 96-well tissue culture plate (Catalog no. #353072, corning incorporated, USA) at a concentration of 10⁵ cells/ml and incubated in 5% CO₂ at 37° C. for 6 hr, followed by washing with DMEM media and cell stimulation.

### HA Digestion for HPLC Analysis and Cell Culture Stimulation

HA (2 mg/ml) from rooster comb was digested with either supernatant from C. acnes bacterial cultures (10 μl/ml) or 1 μg of a purified recombinant protein (rHylA, rHylB or mutant proteins) at a concentration of 0.35 or 1 μl/ml. The digestion was carried out in a reaction buffer containing 100 mM Na acetate, 10 mM CaCl₂ and 0.5 mM DTT (pH=5.5) at 37° C. for different time points (0, 5 min, 15 min, 1 hr and 24 hr), and the reaction was stopped by inactivating the enzyme at 80° C. for 10 min and then stored in −20° C. until further use. Supernatant from bacterial cultures used for HA digestion was 20× concentrated using 50 kDa Amicon® Ultra-15 centrifugal filters (Catalog no. #UFC905024, Millipore Sigma, USA).

For BMDMs and HaCaT cell assays, an equivalent of 40 μg of HA digest was used to stimulate 10⁵ cells for 8 and 16 hr, respectively. Cells were plated in a Falcon® 96-well tissue culture plate and cultured in 200 μl of complete RPMI medium supplemented with 10% FBS and 1× penicillin-streptomycin antibiotics solution. After 8 or 16 hr (or 24 hr) incubation at 37° C. under 5% CO₂, the cells were centrifuged at 400×g and the culture supernatant were collected for analysis of proinflammatory cytokines, including IL-1B, IL-6, TNF-α, and IL-8, by a solid-phase sandwich enzyme-linked immunosorbent assay (ELISA; Biolegend, CA, USA).

HA digested products were analyzed by strong anion exchange high performance liquid chromatography (HPLC), which was performed with the Ultimate 3000 HPLC system (ThermoScientific, USA) equipped with a Ultimate3000 Variable Wavelength Detector on a Pro Pack SAX-10 (4×250 mm) column attached to a Pack SAX-10G guard column (4×50 mm, Thermo-Dionex, USA) at 30° C. Two different solvents were used; Solvent-A (HPLC-water pH 3.5) and Solvent-B (2M NaCl, pH 3.5) at flow rate of 1 mL/min. The gradient conditions (linear) are mentioned in the table below:

| Time(min) | % A | % B |
|---|---|---|
| 2 | 100 | 0 |
| 25 | 75 | 25 |
| 27 | 40 | 60 |
| 32 | 100 | 0 |
| 45 | 100 | 0 |

The chromatogram was acquired with UV absorbance set at 232 nm. Known amount of sample was dissolved in UP water and injected on HPLC. Standard mixture of 1 μg each of HA-DP2, HA-DP4 and HA-DP6 was injected and the HA oligosaccharides in the samples were quantified by comparing the area under the peaks with the standard mixture.

### Phylogeny Analysis

HylA and HylB FASTA amino acid sequences obtained from NCBI or the RCSB Protein data Bank were used for phylogenic analysis. Clustal alignment of sequences was conducted and then, Neighbor-Joining tree was built using the Geneious Prime.

### Expression and Purification of Recombinant Enzymes

C. acnes HylB (residues 37-801) and HylA (41-805) were cloned into pET His6 TEV LIC (Catalog no. #29653, Addgene) and pET His6 MBP TEV LIC (Catalog no. #29656, Addgene) cloning vectors, respectively, and propagated in Escherichia coli Top10 cells (Catalog no. #C404010, ThermoFisher Scientific, USA). The recombinant plasmids were transformed into E. coli BL21 (DE3) pLysS cells (Catalog no. #C606010, ThermoFisher Scientific, USA), and the protein expression was induced by addition of 0.1 mM IPTG (Catalog no. #16758, Sigma-Aldrich, USA) to bacterial cultures (OD-0.6 nm), followed by incubation of cultures at 18° C. for 16 hr. Then bacteria were pelleted at 10000 rpm (or 17700×g) for 10 min and the pellet was resuspended in lysis buffer (50 mM Na₂HPO₄ (e.g., at pH 8), 300 mM NaCl, 2 mM MgCl₂, 10 mM imidazole, 1% Triton X-100, 1 mg/ml egg white lysozyme, 1 mM PMSF, and 10 μg/ml DNases). The bacterial lysate was stored in −80° C. for 24 hr, followed by freeze thawing at 4° C. and centrifugation at 10000 rpm (or 17700×g) for 20 min. The supernatant was harvested and incubated with His60 Ni Superflow™ resin (Catalog no. ##635660, Takara Bio USA, Inc.) for 4 hr, followed by passing the mixture through chromatographic gravity columns. The resin was washed thrice (total 90 ml) with wash buffer (50 mM Na₂HPO₄ (e.g., pH 7.4), 300 mM NaCl and 25 mM imidazole) and the protein was eluted by 15 ml of elution buffer (10 mM Na₂HPO₄ (pH 7.4), 300 mM NaCl, 300 mM imidazole. And 0.1% Tween 80). The eluted protein was washed thrice with PBS-T buffer containing 0.1% tween-80 using 50 kDA Amicon™ centrifugal filters. The purity of purified proteins was confirmed by SDS-PAGE analysis. The purified proteins were cleaned off from LPS contamination using Pierce™ high-capacity endotoxin removal spin columns (Catalog no. #88274, ThermoFisher Scientific, USA) following the instructions of manufacturer.

Mutant hylA and hylB constructs were cloned and expressed with single amino acid substitutions were cloned as above. The concentration of proteins was estimated by NanoDrop 2000 Spectrophotometer (ThermoScientific, USA), and stored in −80° C. until further use.

For protein crystallization studies, BL21 bacterial cells harboring HylA and HylB proteins were harvested by centrifugation at 4000 rpm for 15 min, followed by resuspension in Ni buffer (30 mM HEPES, 500 mM NaCl, 10% glycerol, 20 mM imidazole, 5 mM β-mercaptoethanol; pH 7.5) supplemented by Roche Complete EDTA-free protease inhibitor cocktail. Samples were lysed by sonication, centrifuged at 17,000 rpm (or 23,700×g) for 40 min to remove cellular debris, and applied to a HisTrap FF crude column (GE Healthcare). Protein was eluted using Ni buffer containing 500 mM imidazole. After incubation with TEV protease and dialysis into Ni buffer overnight, samples were again applied to a HisTrap FF crude column to remove uncleaved product. Samples were then purified over a Superdex 200 Increase 10/300 GL column (GE Healthcare) into buffer containing 100 mM Na acetate pH 5, 10 mM CaCl2, and 0.5 mM TCEP, concentrated, and frozen with liquid nitrogen.

### Molecular Dynamics Simulations:

The molecular dynamics simulations were carried out using the GROMACS software package version 2022.4 and as described by Joshi H V et al. Briefly, the HylA-Y285F and HylB-WT apo crystal structures were modeled with missing residues and the residue Phe285 was mutated back to Tyr285. Then two mutant HylA (S452G and E346G) models were generated. Four models (HylB-wt, HylA-wt and HylA-mutants) were used as starting models for simulation studies. A 100 ns MD run was carried out for all four simulations done in this study. Domain motions (Eigenvectors) for each model were determined using PCA analysis from Gromacs package (manual.gromacs.org/2022.4/manual-2022.4). The cleft opening/closing motion (Evec1) was determined as the Cα-Cα separations of Ser97 and Thr636 (HylA numbering); the domain twisting motion (Evec2) as the Cα-Cα separations of Glu208 and Pro216; the substrate-entry opening/closing motion (Evec3) as Cα-Cα separations of Thr80 and Thr636, and the product-exit opening/closing motion (Evec4) as Cα-Cα separations of Thr80 and Thr636.

### Design of the HylA Multi-Epitope Vaccine

Linear B cell epitopes within HylA protein were predicted using Bepipred Linear Epitope Prediction 2.0 (IEDB analysis resources, tools.iedb.org/bcell/). Immunogenic peptides with scores above 0.5 were selected and aligned with HylB. Four peptides with no homology to HylB were selected and physically linked to the C-terminus of a tetanus toxoid protein. Linker amino acid glycine (G) was placed between each peptide and the C-terminus of tetanus toxin (FIG. 15). The fusion gene (TT-mHylA) was then optimized for E. coli protein expression, cloned into pET28a(+) (GenScript), and transformed into E. coli BL21 (DE3) pLysS cells. The HylA multi-epitope construct was then expressed, purified as described above, and assessed for accuracy of sequence by mass spectrometry.

### Crystallization, Data Collection, and Structure Determination

Crystals of HylB were grown using the hanging drop method by adding equal volumes of protein and well solution (0.2 M Na dihydrogen phosphate pH 6.5, 9% PEG 8000, 5 mM TCEP) and suspending over well solution at 18° C. Crystal size and quality were improved using streak seeding. Crystals were cryoprotected in well solution that contained 12% PEG 8000 and 25% glycerol and flash-frozen in liquid nitrogen. Crystals of HylB Y281F were grown as above using well solution 0.1 M bis-tris pH 6.5, 0.4 M MgCl2, 16% PEG 3350, and 5 mM TCEP and cryoprotected in well solution that contained 20% PEG 3350 and 25% glycerol. Crystals of HylA Y285F were grown and cryoprotected as above for HylB except that 0.1 M Na dihydrogen phosphate pH 6.5 was used. Diffraction data were collected on a Rigaku MicroMax-007HF rotating anode X-ray generator with R-Axis IV++ detector.

Diffraction data were processed using XDS, and scaled using Scala. Molecular replacement for HylB was performed using PHASER, with the N-terminal domain of S. agalactiae hyaluronate lyase (PDB: 1F1S) and the C-terminal domain of A. aurescens chondroitin AC lyase (PDB: 1RWA) as search models. The structure of HylB was used as a search model to solve HylB Y281F and HylA Y285F. Model building and refinement were performed using COOT and PHENIX.

After refinement, the Ramachandran statistics for the HylB WT are 97.6% favored, 2.4% allowed, and 0% outliers; while it is 96.82%, 3.11%, and 0.07%, respectively, for HylB Y281F; and 96.5%, 3.37%, and 0.13%, respectively, for HylA Y285F. The structural figures were prepared using the PyMOL visualization tool (The PyMOL Molecular Graphics System, Version 2.4 Schrödinger, LLC.). All the above crystallographic and structure visualization & analysis tools/applications were used on the SBGrid Consortium platform [www.sbgrid.org]. Root mean square deviations between crystal structures of GAG lyases were performed using the Dali Server. The crystal structures and associated data are available from the RCSB Protein Data Bank. The PDB codes for HylA (8FYG [www.rcsb.org/structure/unreleased/8FYG]) and HylB (8FNX [www.rcsb.org/structure/unreleased/8FNX], 8G0O [www.rcsb.org/structure/unreleased/8G0O]).

Pairwise structural comparisons were performed using the Dali Server. The structural figures were prepared using the PyMOL visualization tool.

### Hyaluronidase Enzyme Assay:

Using an Infinite M200 Pro UV spectrophotometer (Tecan), HylA or HylB at concentration 0.0075-0.3 μg/mL and HMW-HA at concentration 0.2 mg/mL in assay buffer were added to a 96-well UV-Star clear microplate (Greiner Bio-One, #655801) with a reaction volume of 100 μl. Reactions were monitored over 10 min at wavelength 232 nm using an Infinite M200 Pro UV spectrophotometer (Tecan). Reaction volume was 100 μL. Assay buffer contained 100 mM Na acetate pH 5.5, 10 mM CaCl₂, and 0.5 mM TCEP. Reaction velocities (absorbance units/sec) were obtained using the slope calculated by Magellan software v. 7.0 over reaction time 1-9.5 minutes. All reactions were performed in triplicate. Enzyme-substrate curves were generated using GraphPad Prism (Ref-Manual) by applying a nonlinear regression fit to the equation for Enzyme kinetics-Michaelis-Menten:

Y=V max*X/(Km+X)

where Y is enzyme velocity in units of absorbance units/sec, X is HMW-HA concentration, Vmax is the maximum enzyme velocity, and Km is the HA-HMW concentration needed to achieve half-maximum enzyme velocity. HMW-HA was hyaluronic acid sodium salt from rooster comb, Sigma #H5388, MW 1-4 million Da. This method was adapted from a previous work.

### Mouse Acne Model

All animal studies were approved under the guidelines of the University of California San Diego (UCSD) Institutional Animal Care and Use Committee. Outbred 6 weeks-old female CD1 mice (The Charles River Laboratory) were housed in an animal facility at UCSD with a standard of care as per federal, state, local, and NIH guidelines.

Six weeks-old C57BL/6, TLR2^{-/-} (Strain #: 004650), and TLR4^{-/-} (Strain #: 004650) mice were purchased from Jackson Laboratories. TLR2^{-/-} and TLR4^{-/-} mice were bred in specific-pathogen free facilities. All mice were provided with sterile food and water ad-libitum, and animal experiments were performed at approximately 8 weeks of age.

To model human acne disease, 8 weeks-old mice were i.d. infected with C. acnes strains (2×10⁷ CFU in 50 μl volume of BHI media), followed by the topical application of synthetic sebum daily as described previously. i.d. infections were performed under vaporized Isoflurane (Fluriso, Vet One) anesthesia. Synthetic sebum was made by mixing fatty acid (17% oleic acid; Catalog no. #01008, Millipore Sigma), triglyceride (45% triolein; Catalog no. #ICN10312201, FisherScientific), wax monoester (25% jojoba oil, Trader Joe), and squalene (13%; Catalog no. #AC215351000, FisherScientific). One or two days after infection, disease score was assessed and the mice were euthanized by CO₂. Skin lesions were aseptically excised and harvested in phosphate buffer saline (PBS, pH 7.4). The skin lesions were then homogenized and 25 μl was serially diluted (10-fold) in PBS to determine CFU on BHI agar plates. The BHI agar plates were incubated anaerobically at 37° C. for 3-4 days. In addition, homogenized skin lesions were centrifuged at maximum speed (13000 rpm) for 20 min and the supernatant was collected and stored in −80° C. for additional analyses.

### Disease Scoring

Gross skin pathology was scored based on tabulation of the following: Erythematous change (no=0, mild=1, moderate 2, and marked=3); papule (flat=0, small=1, large=2. And extra-large=3) based on a protocol modified from.

### Mouse Immunization

Eight-weeks-old CD1 mice were vaccinated i.p. with 200 μl of alum, alum-rHylA, alum-rHylB or alum-tetanus toxoid-multi-epitope HylA fusion protein (TT-mHylA (mEHylA)) at day 1, 7 and 14. Alum-rHylA and Alum-rHylB were prepared by mixing rHylA or rHylB enzyme with 500 μg of Alhydrogel® Alum adjuvant (Catalog no. #vac-alu-50, InvivoGen), followed by gentle rocking on ice for 1 hour. The vaccines were dosed at 70 μg for the first injection and 50 μg for the subsequent two injections. Serum samples were collected seven days after the last vaccination to assess antibody titers against rHylA or rHylB. To assess the protective effect of vaccination against acne, two weeks post-vaccination mice were challenged i.d. with the clinical HL043PA1 or H1110PA3 C. acnes strains (2×10⁷ CFU). Bacterial count (CFU/ml), size of the skin lesions and proinflammatory cytokines were determined on day 1 and 2 post-challenge as previously described.

### Adoptive Transfer of T Cells

Spleens collected on d10 after the last vaccination were homogenized in sterile PBS (pH 7.4), followed by RBC lysis (Catalog number: 00-4300-54, eBioscience™) and isolation of CD3⁺T cells (Catalog No. #480031, Biolegend, USA) by negative selection using MojoSort™ Mouse CD3 T Cell Isolation Kit as per the manufacturer's instructions. 1×10⁷ CD3⁺T cells were retro-orbitally injected into naïve recipient mice. The retroorbital injection was performed under Isoflurane (Fluriso, Vet One) anesthesia. 20 hr post-cell transfer, mice were challenged i.d. with HL043PA1 (2×10⁷ CFU), followed by CFU determination, disease score and skin cytokines on d2 as described above.

### Serum Neutralization of HylA Enzymatic Activity

HylA enzyme (0.3 μg) was incubated at 37° C. for 20 min with 10 μl of pooled serum (n=5), isolated from either mock or mEHylA vaccinated mice. HA (2 mg/ml) was added to the mixture and incubated for 20 hr at 37° C. under continuous rocking. Subsequent HPLC analysis was carried out as mentioned above.

### Determination of Cytokines in Skin Lesions

IL-1β, IL-6, and TNF-α cytokine levels in skin homogenates, previously stored at −80° C., were measured by a solid-phase sandwich ELISA using commercially available mouse cytokine ELISA kits (Biolegend, San Diego, CA, USA). The assay was performed in biological replicates as per manufacturer's instructions. The skin homogenates (50 μl) for IL-1β and IL-6 were diluted 1:1 with the blocking buffer (1% BSA plus 1×PBS-Tween20) and undiluted skin homogenate (100 μl) for TNF-α were used in the assay along with the known concentration of cytokine standards (provided with the kits) in each ELISA plate. The plates were developed and read at optical density (OD) of 450 nm with a wavelength correction set to 570 nm in a multimode microplate reader (PerkinElmer, Waltham, MA, USA). The standard curve generated from the OD of cytokine standards was used to determine cytokine levels in the samples. For determination of cytokine levels in culture supernatants of HaCaT cells, human IL-6 and IL-8 cytokine ELISA kits were purchased from Biolegend. Culture supernatant was diluted 1:1 and the assay were performed as mentioned above.

### Determination of Antibody Titers

Serum antibody titers against rHylA and rHylB were measured by an indirect ELISA method. Briefly, 96-wells high binding microplate (Catalog no. #655081, Greiner Bio-one) were coated overnight at 4° C. with a 500 ng of either rHylA or rHylB in carbonate-bicarbonate buffer (0.2M, pH 9.6). The plates were washed thrice with PBS-T and blocked with 1% BSA (dissolved in PBS-T buffer) for 1 hr at room temperature under continuous rocking. Following washing, the sera samples (1:100, 1:1000, 1:10,000, 1:100,000) diluted in PBS-T were applied to the wells in biological replicates and the plate was incubated for 2 hr at room temperature under continuous rocking. The antibody titers (IgM, IgG, IgG1, IgG2b and IgG3) were detected using goat anti-mouse HRP conjugated antibodies (SouthernBiotech) at 1:5000 dilution in the blocking buffer for 1 hr at room temperature under continuous rocking. After step wash (thrice), the plates were developed at room temperature for 3 min using TMB substrate (Catalog no. #555214, BD Bioscience, CA, USA) and the reaction was stopped by addition of 1N H₂SO₄. The plates were read at optical density (OD) of 450 nm with a wavelength correction set to 570 nm in a multimode microplate reader (PerkinElmer, Waltham, MA, USA).

### Statistical Analysis and Data Reproducibility

GraphPad prism version 8 was used to analyze all data (GraphPad Software, San Diego, CA, graphpad.com). Specific statistical analyses were noted in the figure legends. In vitro experiments were performed independently 2-3 times with at least three technical replicates. Data were presented as mean±standard deviation. In vitro data was analyzed by a non-parametric Mann-Whitney Student's T test and One-way ANOVA. All the in vivo mice data were presented as median of two or more independent experiments. Two-group analysis used a non-parametric Mann-Whitney unpaired Student's T test (two-tailed test). Comparisons of multiple groups were performed using one-way ANOVA with Tuckey's post-hoc test. In the case of missing normality, non-parametric Kruskal-Wallis one-way ANOVA was used to analyze the data

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. An immunogenic polypeptide, comprising:
a fragment of HylA.

2. The immunogenic polypeptide of claim 1, wherein the fragment of HylA is linked or fused to an adjuvant

3. The immunogenic polypeptide of claim 2, wherein the fragment of HylA comprises one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

4. The immunogenic polypeptide of claim 2, wherein the adjuvant is a tetanus protein, pertussis toxoid, diphtheria toxoid, cytokine, or a fragment thereof.

5. A mRNA molecule encoding the immunogenic polypeptide of claim 3.

6. A polypeptide, comprising:
one or more peptides selected from
(a) EMPDAFASPDPDIW (SEQ ID NO:1),
(b) a EMPDAFASPDPDIW (SEQ ID NO:1) variant, the EMPDAFASPDPDIW (SEQ ID NO:1) variant having at least 50% sequence identity to EMPDAFASPDPDIW (SEQ ID NO:1), having up to 7 amino acid additions, substitution, or deletions, or having linear epitope score of at least 0.4, or a combination thereof,
(c) VATILTDLASSSSRTTVLLSANLQKEESS (SEQ ID NO:2),
(d) a VATILTDLASSSSRTTVLLSANLQKEESS (SEQ ID NO:2) variant, the VATILTDLASSSSRTTVLLSANLQKEESS (SEQ ID NO:2) variant having at least 50% sequence identity to VATILTDLASSSSRTTVLLSANLQKEESS (SEQ ID NO:2), having up to 17 amino acid additions, substitution, or deletions, or having linear epitope score of at least 0.4, or a combination thereof,
(e) ENSSDRISVSRS (SEQ ID NO:3),
(f) a ENSSDRISVSRS (SEQ ID NO:3) variant, the ENSSDRISVSRS (SEQ ID NO:3) variant having at least 50% sequence identity to ENSSDRISVSRS (SEQ ID NO:3), having up to 6 amino acid additions, substitution, or deletions, or having linear epitope score of at least 0.4, or a combination thereof,
(g) ALPKPTKPSLRASSYPLGLP (SEQ ID NO:4), or
(h) a ALPKPTKPSLRASSYPLGLP (SEQ ID NO:4) variant, the ALPKPTKPSLRASSYPLGLP (SEQ ID NO:4) variant having at least 50% sequence identity to ALPKPTKPSLRASSYPLGLP (SEQ ID NO:4), having up to 10 amino acid additions, substitution, or deletions, or having linear epitope score of at least 0.4, or a combination thereof.

7. The polypeptide of claim 6, wherein the one or more peptides is linked or fused to an adjuvant.

8. The polypeptide of claim 7, wherein the adjuvant is a tetanus protein, pertussis toxoid, diphtheria toxoid, cytokine, or a fragment thereof.

9. The polypeptide of claim 7, further comprising a linker between the one or more peptides and the adjuvant.

10. The polypeptide of claim 7, further comprising a linker between the one or more peptides and C-terminus of the adjuvant.

11. The polypeptide of claim 6, comprising at least two peptides and further comprising a linker between each of the at least two peptides.

12. The polypeptide of claim 9, wherein the linker is G, polyserine, polyglycine, glycine-serine, GGGGS (SEQ ID NO:5), GGGGGS (SEQ ID NO:6), leucine zipper, r aliphatic, or helical peptides.

13. A composition comprising a polypeptide claim 6, and an adjuvant.

14. The composition of claim 13, wherein the adjuvant is alum, hydroxyphosphate sulfate, CpG1018, monophosphoryl lipid A, oil-in-water emulsion, CpG, or QS-21 saponin.

15. A mRNA molecule encoding the polypeptide of claim 6.

16. A method of treating or reducing the likelihood of acne, comprising: administering a polypeptide of claim 1 to a subject in need thereof.

17. A method of treating or reducing the likelihood of acne, comprising: administering a polypeptide of claim 6 to a subject in need thereof.

18. A method of treating or reducing the likelihood of acne, comprising: administering a composition of claim 13 to a subject in need thereof.

19. A method of treating or reducing the likelihood of acne, comprising: administering a composition comprising the mRNA molecule of claim 5 to a subject in need thereof.

20. A method of treating or reducing the likelihood of acne, comprising: administering a composition comprising the mRNA molecule of claim 15 to a subject in need thereof.
